(19) United States
(12) Patent Application Publication
(10) Pub. No.: US 2024/0156994 A1
(43) Pub. Date: May 16, 2024

(54) FLUORINE-CONTAINING COMPOUND AND CONTRAST MEDIUM

(71) Applicant: TDK CORPORATION, Tokyo (JP)

(72) Inventor: Naoko YANAI, Tokyo (JP)

(73) Assignee: TDK CORPORATION, Tokyo (JP)

(21) Appl. No.: 18/280,304

(22) PCT Filed: Mar. 16, 2022
(86) PCT No.: PCT/JP2022/011975

§ 371 Date: Sep. 5, 2023

(30) Foreign Application Priority Data
Mar. 17, 2021 (JP) 2021-043724

(51) Int. Cl. A61K 49/10; C07D 207/46

(52) U.S. Cl. CPC A61K 49/10; C07D 207/46

(57) ABSTRACT

A fluorine-containing compound represented by Formula (1) or Formula (2) (R¹, R², and R³ in Formula (1) and R⁴ and R⁵ in Formula (2) each represent a C1-10 alkyl group unsubstituted or substituted with a substituent containing no fluorine atoms, and X¹ in Formula (1) and X² and X³ in Formula (2) are any of Formulae (3-1) to (3-4).)

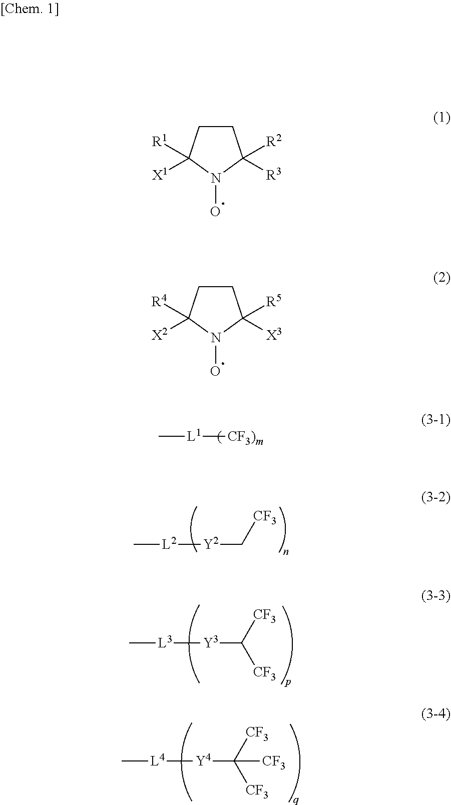

### TECHNICAL FIELD

The present invention relates to a fluorine-containing compound and a contrast medium.

Priority is claimed on Japanese Patent Application No. 2021-043724, filed on Mar. 17, 2021, the content of which is incorporated herein by reference.

### BACKGROUND ART

Magnetic resonance imaging (hereinafter sometimes referred to as “MRI”) diagnosis is widely used in the medical field for both basic research and clinical application as one of diagnostic imaging methods along with X-ray diagnosis and ultrasound (US) diagnosis.

Currently, ¹H-MRI using protons (¹H) as detection nuclei is used for medical MRI. ¹H-MRI captures and images the magnetic environment of water molecules present in vivo. A difference occurs in the magnetic environment of protons between lesion tissue and normal tissue in vivo. This appears as a difference in ¹H-MRI and serves as diagnostic information. In addition, water molecules are present almost everywhere in vivo. For this reason, ¹H-MRI can be used for whole-body imaging.

Nuclides detectable by MRI include ¹⁹F, ²³Na, ³¹P, ¹⁵N, ¹³C, and the like in addition to ¹H. MRI using these elements as detection nuclei provides information different from ¹H-MRI.

Among these, MRI using ¹⁹F as a detection nucleus is expected to be used as a next-generation diagnostic method following ¹H-MRI diagnosis. This is because fluorine is an inexpensive element with a natural abundance ratio of 100%, the detection sensitivity of ¹⁹F is as high as 83% of that of ¹H, and the gyromagnetic ratio of ¹⁹F is close to that of a proton, and therefore, imaging can be performed with a conventional ¹H-MRI device.

In addition, ¹⁹F detectable by MRI is almost non-existent in vivo. For this reason, by using a fluorine atom-containing compound in a contrast medium, ¹⁹F-MRI diagnosis using ¹⁹F as a tracer is possible. For example, positional information of lesion portions can be obtained from ¹⁹F-MRI using a fluorine compound, which recognizes and accumulates endogenous changes caused by a disease, in a contrast medium. This method is useful for diagnosing lesion portions that do not cause morphological changes that could not be detected by conventional diagnostic imaging methods.

Currently, there is a nuclear medicine technique as a method of obtaining image information specific to a lesion portion. Nuclear medicine techniques use radiopharmaceuticals in which radioisotopes are used. Specifically, nuclear medicine techniques include a positron emission tomography (PET) examination, and a single photon emission computed tomography (SPECT) examination. However, the nuclear medicine techniques have problems such as large-scale apparatuses for synthesizing radioisotopes and a risk of radiation exposure.

¹⁹F-MRI diagnosis does not cause the above-described problems in nuclear medicine techniques. In addition, in ¹⁹F-MRI diagnosis, by extracting information such as chemical shift, diffusion, and relaxation time, more diagnostic information can be obtained in addition to the positional information of the lesion portions. In addition, by taking ¹⁹F-MRI and ¹H-MRI simultaneously in one diagnosis and superimposing the images, it is possible to obtain useful diagnostic information in which anatomical information and functional information coexist.

Contrast media for MRI diagnosis using fluorine as a detection nucleus are disclosed, for example, in Patent Documents 1 and 2.

Patent Document 1 discloses lactic acid-glycolic acid copolymer (PLGA) particles containing perfluoro crown ether and gadolinium complexes. In addition, Patent Document 2 discloses a fluorine-containing porphyrin complex and a contrast medium compound which can be used in MRI using fluorine as a detection nucleus.

However, since the contrast media disclosed in Patent Documents 1 and 2 contain metal ions, there is a concern about their safety in vivo.

In addition, Patent Document 3 discloses a compound having a nitroxide covalently bound to a fluorine-containing compound. However, since the nitroxide compound is easily reduced by a reducing agent such as ascorbic acid (for example, refer to Non-Patent Document 1), it has a problem of stability in vivo.

### CITATION LIST

### Patent Documents

[Patent Document 1]
Published Japanese Translation No. 2015-534549 of the PCT International Publication

[Patent Document 2]
Japanese Unexamined Patent Application, First Publication No. H11-217385

[Patent Document 3]
U.S. Pat. No. 5,362,477(B)

### Non-Patent Document

[Non-Patent Document 1]
ACS Central Science, 2017, 3, 800-811

### SUMMARY OF INVENTION

### Technical Problem

The conventional contrast media for MRI diagnosis using fluorine as a detection nucleus do not provide high-sensitivity MRI and are not highly stable in vivo.

The present invention has been made in consideration of the above-described circumstances, and an object of the invention is to provide a fluorine-containing compound which is used as a material for a contrast medium for magnetic resonance imaging diagnosis using fluorine as a detection nucleus and is highly stable in vivo and from which a high-sensitivity magnetic resonance image is obtained.

In addition, another object of the present invention is to provide a contrast medium for magnetic resonance imaging diagnosis using fluorine as a detection nucleus which contains the fluorine-containing compound of the present invention and is highly stable in vivo and from which a high-sensitivity image can be obtained.

### Solution to Problem

[1] A fluorine-containing compound represented by General Formula (1) or General Formula (2) below.

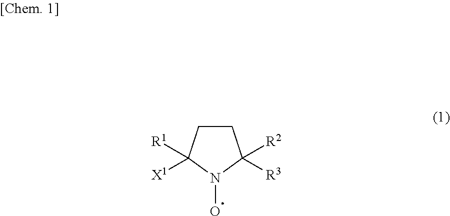

(In General Formula (1), R¹, R², and R³ each independently represent a C1-10 alkyl group unsubstituted or substituted with a substituent containing no fluorine atoms, and X¹ is a substituent represented by any of General Formulae (3-1) to (3-4).)

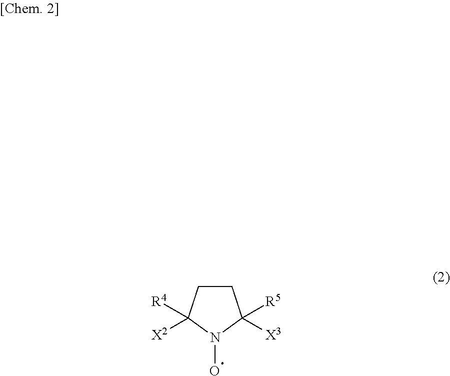

(In General Formula (2), R⁴ and R⁵ each independently represent a C1-10 alkyl group unsubstituted or substituted with a substituent containing no fluorine atoms, and X² and X³ are each independently a substituent represented by any of General Formulae (3-1) to (3-4).)

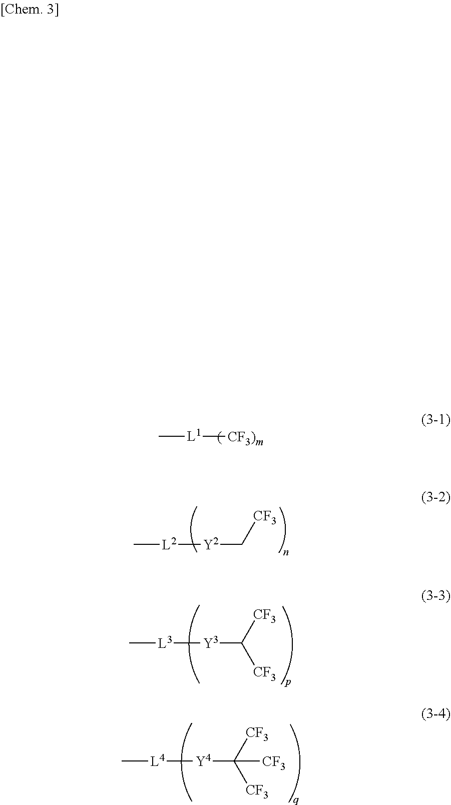

(In General Formula (3-1), L¹ is either a C1-10 chain hydrocarbon group unsubstituted or substituted with a substituent containing no fluorine atoms or a linking group containing a C6-12 aryl group unsubstituted or substituted with a substituent containing no fluorine atoms, and m is an integer of 1 to 5.)

(In General Formula (3-2), L² is either a C1-10 chain hydrocarbon group unsubstituted or substituted with a substituent containing no fluorine atoms or a linking group containing a C6-12 aryl group unsubstituted or substituted with a substituent containing no fluorine atoms, Y² is an oxygen atom, and n is an integer of 1 to 5.)

(In General Formula (3-3), L³ is either a C1-10 chain hydrocarbon group unsubstituted or substituted with a substituent containing no fluorine atoms or a linking group containing a C6-12 aryl group unsubstituted or substituted with a substituent containing no fluorine atoms, Y³ is an oxygen atom, and p is an integer of 1 to 5.)

(In General Formula (3-4), L⁴ is either a C1-10 chain hydrocarbon group unsubstituted or substituted with a substituent containing no fluorine atoms or a linking group containing a C6-12 aryl group unsubstituted or substituted with a substituent containing no fluorine atoms, Y⁴ is an oxygen atom, and q is an integer of 1 to 5.)

[2] The fluorine-containing compound according to [1], in which R¹, R², and R³ in General Formula (1) above each independently represent a C1-5 alkyl group unsubstituted or substituted with a substituent containing no fluorine atoms.

[3] The fluorine-containing compound according to [1], in which R⁴ and R⁵ in General Formula (2) above each independently represent a C1-5 alkyl group unsubstituted or substituted with a substituent containing no fluorine atoms.

[4] The fluorine-containing compound according to [1] or [3], in which, in General Formula (2) above, X² is the same as X³ and R⁴ is the same as R⁵.

[5] The fluorine-containing compound according to any one of [1] to [4], in which L¹ in General Formula (3-1), L² in General Formula (3-2), L³ in General Formula (3-3), and L⁴ in General Formula (3-4) are C1-5 chain hydrocarbon groups unsubstituted or substituted with a substituent containing no fluorine atoms.

[6] The fluorine-containing compound according to any one of [1] to [4], in which L¹ in General Formula (3-1), L² in General Formula (3-2), L³ in General Formula (3-3), and L⁴ in General Formula (3-4) are each a linking group containing a phenyl group or a biphenyl group.

[7] The fluorine-containing compound according to any one of [1] to [6], in which m in General Formula (3-1), n in General Formula (3-2), p in General Formula (3-3), and q in General Formula (3-4) are each 1 or 2.

[8] The fluorine-containing compound according to any one of [1] to [7], which is used in a contrast medium for magnetic resonance imaging diagnosis using fluorine as a detection nucleus.

[9] A contrast medium for magnetic resonance imaging diagnosis using fluorine as a detection nucleus, including: the fluorine-containing compound according to any one of [1] to [8].

### Advantageous Effects of Invention

The fluorine-containing compound of the present invention is a compound represented by General Formula (1) and General Formula (2) above. For this reason, it is highly stable in vivo. In addition, the fluorine-containing compound of the present invention is used as a material for a contrast medium for magnetic resonance imaging diagnosis using fluorine as a detection nucleus to obtain a high-sensitivity magnetic resonance image.

A contrast medium of the present invention contains the fluorine-containing compound of the present invention. For this reason, the contrast medium of the present invention is highly stable in vivo. In addition, the contrast medium of the present invention is used to obtain a high-sensitivity magnetic resonance image using fluorine as a detection nucleus.

### BRIEF DESCRIPTION OF DRAWINGS

FIG. 1 is a ¹⁹F spin-lattice relaxation time (T1)-weighted ¹⁹F-MRI image for Example 4 (compound 14).

FIG. 2 is a ¹⁹F spin-lattice relaxation time (T1)-weighted ¹⁹F-MRI image for Example 6 (compound 16).

FIG. 3 is a ¹⁹F spin-lattice relaxation time (T1)-weighted ¹⁹F-MRI image for Comparative Example 1 (compound A1).

### DESCRIPTION OF EMBODIMENT

Hereinafter, a fluorine-containing compound and a contrast medium of the present invention will be described in detail.

### [Fluorine-Containing Compound]

A fluorine-containing compound of the present embodiment is represented by General Formula (1) or General Formula (2) below.

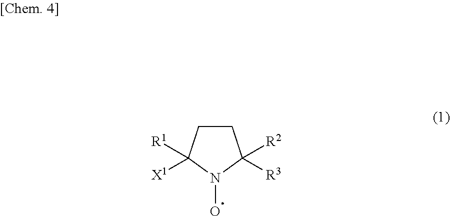

(In General Formula (1), R¹, R², and R³ each independently represent a C1-10 alkyl group unsubstituted or substituted with a substituent containing no fluorine atoms, and X¹ is a substituent represented by any of General Formulae (3-1) to (3-4).)

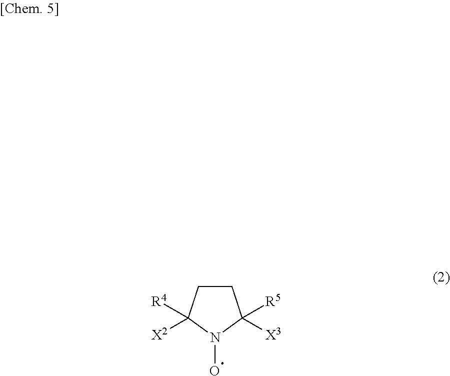

(In General Formula (2), R⁴ and R⁵ each independently represent a C1-10 alkyl group unsubstituted or substituted with a substituent containing no fluorine atoms, and X² and X³ are each independently a substituent represented by any of General Formulae (3-1) to (3-4).)

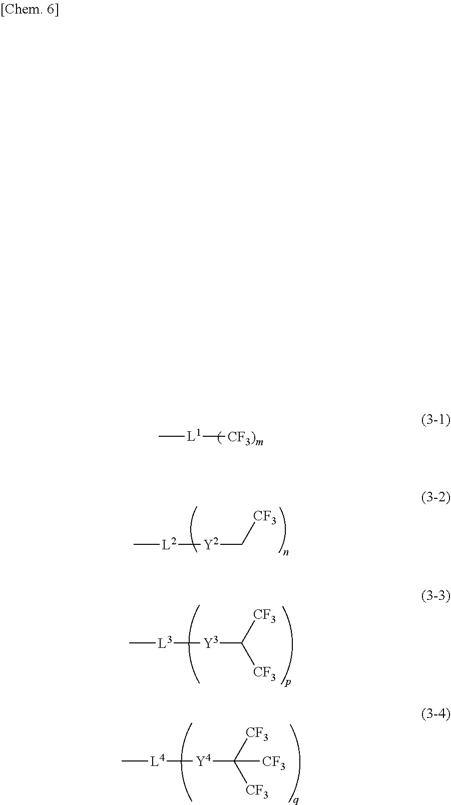

(In General Formula (3-1), L¹ is either a C1-10 chain hydrocarbon group unsubstituted or substituted with a substituent containing no fluorine atoms or a linking group containing a C6-12 aryl group unsubstituted or substituted with a substituent containing no fluorine atoms, and m is an integer of 1 to 5.)

(In General Formula (3-2), L² is either a C1-10 chain hydrocarbon group unsubstituted or substituted with a substituent containing no fluorine atoms or a linking group containing a C6-12 aryl group unsubstituted or substituted with a substituent containing no fluorine atoms, Y² is an oxygen atom, and n is an integer of 1 to 5.)

(In General Formula (3-3), L³ is either a C1-10 chain hydrocarbon group unsubstituted or substituted with a substituent containing no fluorine atoms or a linking group containing a C6-12 aryl group unsubstituted or substituted with a substituent containing no fluorine atoms, Y³ is an oxygen atom, and p is an integer of 1 to 5.)

(In General Formula (3-4), L⁴ is either a C1-10 chain hydrocarbon group unsubstituted or substituted with a substituent containing no fluorine atoms or a linking group containing a C6-12 aryl group unsubstituted or substituted with a substituent containing no fluorine atoms, Y⁴ is an oxygen atom, and q is an integer of 1 to 5.)

Here, the reason why the contrast medium containing the fluorine-containing compound of the present embodiment is highly stable in vivo and a high-sensitivity magnetic resonance imaging (MRI) can be obtained when the contrast medium is used as a contrast medium for MRI diagnosis using fluorine as a detection nucleus will be described.

In order to obtain high-sensitivity ¹⁹F-MRI, it is preferable to use a fluorine-containing compound with a short ¹⁹F spin-lattice relaxation time (T1) as a fluorine-containing compound contained in a contrast medium. The shorter the T1 of the fluorine-containing compound, the shorter the repetition time (TR) can be set. For this reason, the amount of signal obtained per unit time is increased, and a high-sensitivity image can be obtained. On the other hand, if the ¹⁹F spin-spin relaxation time (T2) of the fluorine-containing compound is too short, the signal intensity will decrease.

The ¹⁹F spin-lattice relaxation time (T1) and ¹⁹F spin-spin relaxation time (T2) of a fluorine-containing compound are affected by a paramagnetic relaxation enhancement (PRE) effect. The PRE effect is a phenomenon in which T1 and T2 of MRI observation nuclei in the vicinity of unpaired electron spins possessed by a paramagnetic material are shortened by the unpaired electron spins.

The PRE effect is inversely proportional to the sixth power of the distance between a paramagnetic substance and a MRI observation nucleus (a fluorine atom in the present embodiment) relaxed by the paramagnetic material. Accordingly, in the fluorine-containing compound represented by Formula (1) or Formula (2) of the present embodiment, the shorter the distance between the fluorine atom and a nitroxide radical which is a paramagnetic material, the shorter T1 and T2 are. In the fluorine-containing compound represented by Formula (1) or Formula (2), substituents (X¹ in Formula (1) and X² and X³ in Formula (2)) to which the fluorine atom is bound at a terminal are bound to carbon atoms at the 2- and/or 5-position of the pyrrolidine ring. For this reason, the distance between the nitroxide radical and the fluorine atom is appropriate, T1 is sufficiently short, and T2 can be sufficiently secured. Accordingly, the fluorine-containing compound represented by Formula (1) or Formula (2) is used in a contrast medium for MRI diagnosis using fluorine as a detection nucleus to obtain a high-sensitivity magnetic resonance image.

In addition, unlike closed-shell species, an organic radical has a semi-occupied molecular orbital (SOMO) with unpaired electrons between the highest occupied molecular orbital (HOMO) and the lowest unoccupied molecular orbital (LUMO). A redox process of organic radicals corresponds to an electron transfer process in a SOMO. The reduction reaction of organic radicals due to a reducing agent such as ascorbic acid is more likely to occur when the energy difference between the HOMO of the reducing agent and the SOMO of the organic radicals is smaller. Accordingly, the lower the SOMO energy level of the organic radicals, the easier they are to reduce.

In the fluorine-containing compound represented by Formula (1) or Formula (2) of the present embodiment, the carbon atoms at the 2- and/or 5-position of the pyrrolidine ring are bound to the fluorine atoms contained in the substituent represented by any of Formulae (3-1) to (3-4) via a linking group in which two or more carbon atoms are linked. Accordingly, in the fluorine-containing compound represented by Formula (1) or Formula (2), the nitroxide radical and the fluorine atom are arranged at sufficiently distant positions, and therefore the nitroxide radical is less susceptible to electronic influence from the fluorine atom. Accordingly, in the fluorine-containing compound represented by Formula (1) or Formula (2), the SOMO energy level of the nitroxide radical does not decrease due to the fluorine atom which is an electron withdrawing group. Accordingly, the SOMO of the nitroxide radical in the fluorine-containing compound of the present embodiment has a sufficiently large energy difference from the HOMO of the reducing agent such as ascorbic acid. Accordingly, the fluorine-containing compound represented by Formula (1) or Formula (2) is less likely to be reduced in vivo and highly stable in vivo.

On the other hand, in a case of, for example, a fluorine-containing compound represented by Formula (1) or Formula (2) in which trifluoromethyl groups are bound to carbon atoms at the 2- and/or 5-position of the pyrrolidine ring, the compound is likely to be reduced in vivo due to reasons shown below. That is, in this fluorine-containing compound, there is only one carbon atom between a fluorine atom and carbon atoms at the 2- and/or 5-position of the pyrrolidine ring, and therefore, the distance between the nitroxide radical and the fluorine atom is short. For this reason, the nitroxide radical contained in the fluorine-containing compound is susceptible to electronic influence from the fluorine atom, and therefore, the SOMO energy level would be lowered due to the effect of the fluorine atom as an electron withdrawing group. As a result, the fluorine-containing compound represented by Formula (1) or Formula (2) in which trifluoromethyl groups are bound to carbon atoms at the 2- and/or 5-position of the pyrrolidine ring has a small energy difference between the SOMO of the nitroxide radicals and the HOMO of the reducing agent. Accordingly, this fluorine-containing compound is more likely to be reduced in vivo compared with the fluorine-containing compound represented by Formula (1) or Formula (2) of the present embodiment.

In addition, in the fluorine-containing compound represented by Formula (1) or Formula (2), the substituents represented by any of Formulae (3-1) to (3-4) which are three-dimensionally bulky are bound to the carbon atoms at the 2- and/or 5-position of the pyrrolidine ring, and substituents (R¹, R², and R³ in Formula (1) and R⁴ and R⁵ in Formula (2)) are bound thereto. Accordingly, in the fluorine-containing compound represented by Formula (1) or Formula (2), the approach of a reducing agent to nitroxide radicals is three-dimensionally blocked and hindered by the substituents represented by any of Formulae (3-1) to (3-4) and R¹, R², and R³ or R⁴ and R⁵.

Thus, the fluorine-containing compound represented by Formula (1) or Formula (2) is less likely to be reduced in vivo and highly stable in vivo.

Moreover, since the fluorine-containing compound represented by Formula (1) or Formula (2) of the present embodiment is a non-metal compound containing no metal, it is highly safer in vivo compared with a contrast medium containing metal ions. Accordingly, the fluorine-containing compound of the present embodiment is suitable as a material for a contrast medium for magnetic resonance imaging diagnosis using fluorine as a detection nucleus.

R¹, R², and R³ in the fluorine-containing compound represented by Formula (1) of the present embodiment are each independently a C1-10 alkyl group unsubstituted or substituted with a substituent containing no fluorine atoms and preferably a C1-5 alkyl group unsubstituted or substituted with a substituent containing no fluorine atoms. If R¹, R², and R³ are substituted or unsubstituted C1-10 alkyl groups, they become appropriately bulky and can prevent the approach of a reducing agent to nitroxide radicals. Since the number of carbon atoms in the above-described alkyl group is 10 or less, the fluorine-containing compound represented by Formula (1) is easily synthesized. If the number of carbon atoms in the above-described alkyl group is 5 or less, synthesis of the fluorine-containing compound represented by Formula (1) becomes much easier, which is preferable.

In a case where R¹, R², and R³ contained in the fluorine-containing compound represented by Formula (1) have a substituent containing no fluorine atoms, a methyl group, an ethyl group, or a phenyl group can be used as the substituent, for example.

Specifically, R¹, R², and R³ in the fluorine-containing compound represented by Formula (1) of the present embodiment are preferably a methyl group or an ethyl group, and more preferably a methyl group for easy synthesis.

In the fluorine-containing compound represented by Formula (1) of the present embodiment, X¹ represents a substituent represented by any of Formulae (3-1) to (3-4). For this reason, in the fluorine-containing compound represented by Formula (1), the distance between the nitroxide radical and the fluorine atom is appropriate, T1 is sufficiently short, and T2 can be sufficiently secured. Accordingly, the fluorine-containing compound represented by Formula (1) is used in a contrast medium for MRI diagnosis using fluorine as a detection nucleus to obtain a high-sensitivity image. In addition, in the fluorine-containing compound represented by Formula (1), carbon at the 2-position of the pyrrolidine ring is bound to a fluorine atom via a linking group to which two or more carbon atoms are linked. Accordingly, it is less susceptible to electronic influence from the fluorine atom. Moreover, since the substituents represented by Formulae (3-1) to (3-4) are all bulky, the approach of a reducing agent to the nitroxide radical is three-dimensionally blocked and prevented. Accordingly, the fluorine-containing compound represented by Formula (1) is less likely to be reduced in vivo and highly stable in vivo.

Similarly to R¹, R², and R³ in the fluorine-containing compound represented by Formula (1), R⁴ and R⁵ in the fluorine-containing compound represented by Formula (2) of the present embodiment are each independently a C1-10 alkyl group unsubstituted or substituted with a substituent containing no fluorine atoms, preferably a C1-5 alkyl group unsubstituted or substituted with a substituent containing no fluorine atoms, and more preferably a methyl group or an ethyl group.

Similarly to X¹ in the fluorine-containing compound represented by Formula (1), X² and X³ in the fluorine-containing compound represented by Formula (2) of the present embodiment each represent a substituent represented by any of Formulae (3-1) to (3-4).

In the fluorine-containing compound represented by Formula (2) of the present embodiment, the substituent represented by any of Formulae (3-1) to (3-4) is bound to the 2-position and the 5-position of the pyrrolidine ring, and therefore, the approach of a reducing agent to nitroxide radicals is hindered, making the compound even more difficult to be reduced. In addition, when the fluorine-containing compound represented by Formula (2) contains more fluorine atoms compared with the fluorine-containing compound represented by Formula (1), a higher ¹⁹F-MRI signal can be obtained.

In the fluorine-containing compound represented by Formula (2) of the present embodiment, it is preferable that X² be the same as X³ and R⁴ be the same as R⁵. In a case where such a fluorine-containing compound is used as a contrast medium for magnetic resonance imaging diagnosis using fluorine as a detection nucleus, a single ¹⁹F-MRI peak is exhibited. For this reason, high-quality ¹⁹F-MRI in which chemical shift artifacts are suppressed is obtained.

In the substituent represented by Formula (3-1) contained in the fluorine-containing compound represented by Formula (1) or Formula (2), L¹ is either a C1-10 chain hydrocarbon group unsubstituted or substituted with a substituent containing no fluorine atoms or a linking group containing a C6-12 aryl group unsubstituted or substituted with a substituent containing no fluorine atoms.

If L¹ is a C1-10 chain hydrocarbon group unsubstituted or substituted with a substituent containing no fluorine atoms, the distance between the nitroxide radical and the fluorine atom is appropriate. In the case where L¹ is a C1-10 chain hydrocarbon group unsubstituted or substituted with a substituent containing no fluorine atoms, a C1-5 chain hydrocarbon group unsubstituted or substituted with a substituent containing no fluorine atoms is preferable. If the above-described chain hydrocarbon group has 10 or less carbon atoms, the distance between the nitroxide radical and the fluorine atom does not become too long, and T1 is sufficiently short. If the above-described chain hydrocarbon group has 5 or less carbon atoms, T1 becomes shorter, which is preferable.

In a case where the C1-10 chain hydrocarbon group which is unsubstituted or substituted with a substituent containing no fluorine atoms and represented by L¹ has a substituent, a substituent, such as a methyl group, an ethyl group, or a phenyl group, containing no fluorine atoms can be used.

In the case where L¹ is a C1-10 chain hydrocarbon group unsubstituted or substituted with a substituent containing no fluorine atoms, —(CH₂)₂— or —(CH₂)₃— is more preferable. In this case, the distance between the nitroxide radical and the fluorine atom is more appropriate. As a result, a fluorine-containing compound in which the nitroxide radical is less susceptible to electronic influence from the fluorine atom and which has a high in vivo stability is obtained. Moreover, since this fluorine-containing compound has a shorter T1, in a case where this is used as a contrast medium for MRI diagnosis using fluorine as a detection nucleus, a higher-sensitivity image is obtained.

If L¹ is a linking group containing a C6-12 aryl group unsubstituted or substituted with a substituent containing no fluorine atoms, the distance between the nitroxide radical and the fluorine atom is appropriate. In the case where L¹ is a linking group containing a C6-12 aryl group unsubstituted or substituted with a substituent containing no fluorine atoms, a linking group containing a phenyl group or a biphenyl group is preferable. If the above-described aryl group has 12 or less carbon atoms, the distance between the nitroxide radical and the fluorine atom does not become too long, and T1 is sufficiently short. In the case where L¹ is a linking group containing a phenyl group or a biphenyl group, the fluorine-containing compound represented by Formula (1) or Formula (2) is easily synthesized, which is preferable.

In a case where the Linking group containing a C6-12 aryl group which is unsubstituted or substituted with a substituent containing no fluorine atoms and represented by L¹ has a substituent, a substituent, such as a methyl group, an ethyl group, or a phenyl group, containing no fluorine atoms can be used.

In the case where L¹ is a linking group containing a C6-12 aryl group unsubstituted or substituted with a substituent containing no fluorine atoms, any one selected from a p-phenylene group, a m-phenylene group, and an o-phenylene group is preferable. In this case, the distance between the nitroxide radical and the fluorine atom is more appropriate, and L¹ is bulky. As a result, a fluorine-containing compound in which the nitroxide radical is less susceptible to electronic influence from the fluorine atom and which has a high in vivo stability is obtained. Furthermore, since this fluorine-containing compound has a sufficiently short T1, in a case where this is used as a contrast medium for MRI diagnosis using fluorine as a detection nucleus, a higher-sensitivity image is obtained.

In the substituent represented by Formula (3-1) contained in the fluorine-containing compound represented by Formula (1) or Formula (2), m is an integer of 1 to 5.

In the substituent represented by Formula (3-1), in the case where L¹ is a C1-10 chain hydrocarbon group unsubstituted or substituted with a substituent containing no fluorine atoms, m is an integer of 1 to 3, preferably 1 or 2, and most preferably 1. In a case where L¹ is a C1-10 chain hydrocarbon group unsubstituted or substituted with a substituent containing no fluorine atoms and the fluorine-containing compound in which m is 1 to 3 is used as a contrast medium for magnetic resonance imaging diagnosis using fluorine as a detection nucleus, a single ¹⁹F-MRI peak is exhibited. For this reason, high-quality ¹⁹F-MRI in which chemical shift artifacts are suppressed is obtained.

In the case where L¹ is a linking group containing a C6-12 aryl group unsubstituted or substituted with a substituent containing no fluorine atoms, m is an integer of 1 to 5, preferably 1 or 2, and most preferably 1. In the case where a fluorine-containing compound in which L¹ is a linking group containing a C6-12 aryl group unsubstituted or substituted with a substituent containing no fluorine atoms and m is 1 or 2 is used as a contrast medium for magnetic resonance imaging diagnosis using fluorine as a detection nucleus, a single ¹⁹F-MRI peak is exhibited. For this reason, high-quality ¹⁹F-MRI in which chemical shift artifacts are suppressed is obtained.

L² in Formula (3-2), L³ in Formula (3-3), and L⁴ in General Formula (3-4) contained in the fluorine-containing compound represented by Formula (1) or Formula (2) are, similarly to L¹ in Formula (3-1), each independently either a C1-10 chain hydrocarbon group unsubstituted or substituted with a substituent containing no fluorine atoms or a linking group containing a C6-12 aryl group unsubstituted or substituted with a substituent containing no fluorine atoms.

In a case where L² in Formula (3-2), L³ in Formula (3-3), and L⁴ in General Formula (3-4) are a C1-10 chain hydrocarbon group unsubstituted or substituted with a substituent containing no fluorine atoms similarly to L¹ in Formula (3-1), these are preferably a C1-5 chain hydrocarbon group unsubstituted or substituted with a substituent containing no fluorine atoms and more preferably —(CH₂)₂— or —(CH₂)₃—.

In a case where L² in Formula (3-2), L³ in Formula (3-3), and L⁴ in General Formula (3-4) are a linking group containing a C6-12 aryl group unsubstituted or substituted with a substituent containing no fluorine atoms similarly to L¹ in Formula (3-1), these are preferably a linking group containing a phenyl group or a biphenyl group and more preferably any one selected from a p-phenylene group, a m-phenylene group, and an o-phenylene group.

In the substituent represented by Formula (3-2) contained in the fluorine-containing compound represented by Formula (1) or Formula (2), Y² is an oxygen atom (ether bond).

In the case where L² is a C1-10 chain hydrocarbon group unsubstituted or substituted with a substituent containing no fluorine atoms, Y² is bound to the most terminal carbon atom among carbon atoms in the above-described chain hydrocarbon group.

In the case where L² is a linking group containing a C6-12 aryl group unsubstituted or substituted with a substituent containing no fluorine atoms, Y² is bound through the most terminal aryl group among aryl groups in the above-described linking group.

Y³ in Formula (3-3) and Y⁴ in General Formula (3-4) contained in the fluorine-containing compound represented by Formula (1) or Formula (2) are oxygen atoms similarly to Y² in Formula (3-2).

n in Formula (3-2), p in Formula (3-3), and q in Formula (3-4) contained in the fluorine-containing compound represented by Formula (1) or Formula (2) are each independently an integer of 1 to 5 similarly to m in Formula (3-1), and are preferably 1 or 2.

Specifically, the fluorine-containing compound represented by Formula (1) or Formula (2) is preferably any one of fluorine-containing compounds represented by Formulae (11) to (26) below.

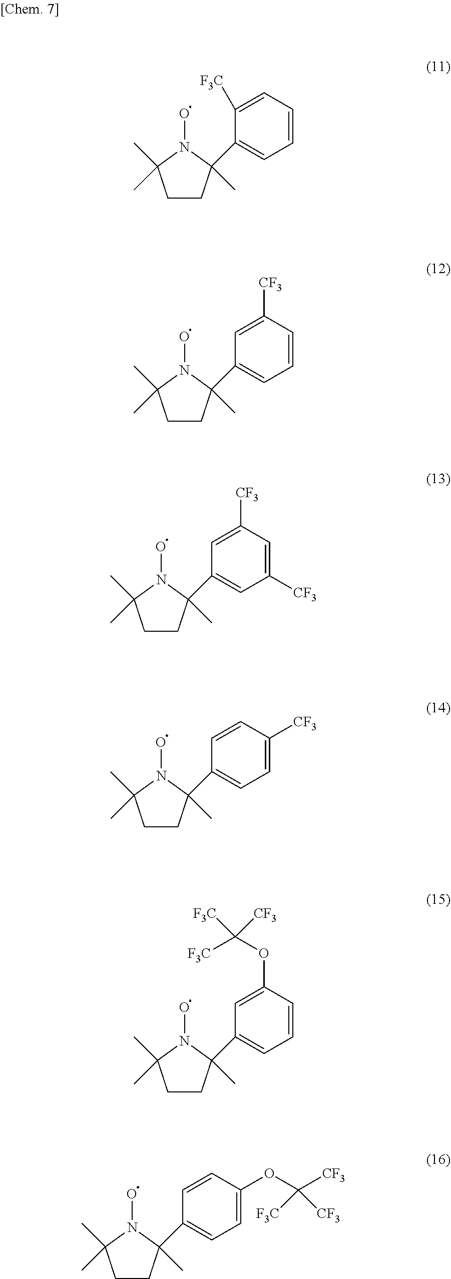

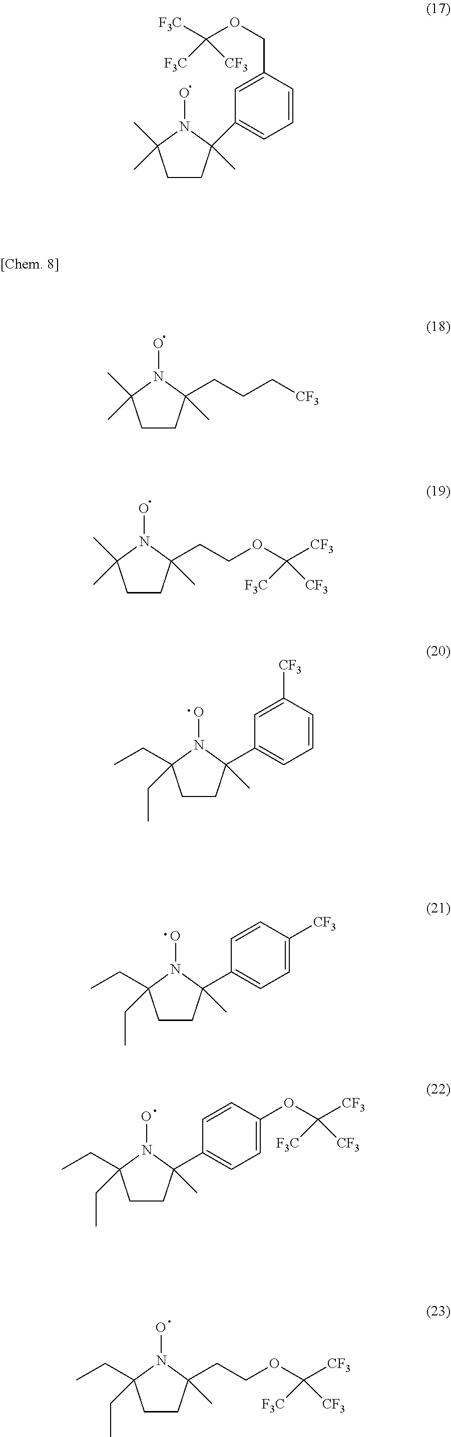

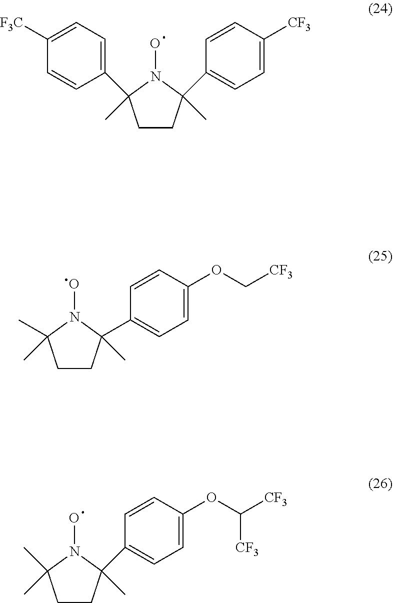

### [Method for Producing Fluorine-Containing Compound]

Next, a method for producing a fluorine-containing compound of the present embodiment represented by Formula (1) or Formula (2) will be described as an example.

The method for producing a fluorine-containing compound of the present embodiment is not particularly limited, and the fluorine-containing compound can be produced using a well-known conventional production method.

The fluorine-containing compound of the present embodiment represented by Formula (1) can be produced using, for example, a production method shown below.

First, a first intermediate consisting of 3,4-dihydro-2H-pyrrole-1-oxide in which R² and R³ are bound to the 2-position and R¹ is bound to the 5-position in the fluorine-containing compound represented by Formula (1) is synthesized. In addition, a Grignard reagent having a group corresponding to X¹ in the fluorine-containing compound represented by Formula (1) is prepared. Then, the first intermediate compound and the Grignard reagent having a group corresponding to X¹ are subjected to a Grignard reaction to introduce X¹ in the fluorine-containing compound represented by Formula (1) into the 5-position of the pyrrole ring and synthesize a second intermediate compound in which a hydroxyl group is bound to a nitrogen atom at the 1-position.

The second intermediate compound may be synthesized through a method shown below. That is, a fluorine-containing compound having a group corresponding to R², R³, and X¹ in the fluorine-containing compound represented by Formula (1) is synthesized. Next, this fluorine-containing compound is cyclized to synthesize a compound having a pyrrole ring skeleton. Then, the resulting compound having a pyrrole ring skeleton and a Grignard reagent having a group corresponding to R are subjected to a Grignard reaction to introduce R¹ in the fluorine-containing compound represented by Formula (1) into the 5-position of the pyrrole ring and synthesize a second intermediate compound in which a hydroxyl group is bound to a nitrogen atom at the 1-position.

Thereafter, the hydroxyl group bound to the nitrogen atom at the 1-position of the pyrrole ring of the second intermediate compound is converted into a nitroxide radical placed at the 1-position of the pyrrolidine ring.

The fluorine-containing compound represented by Formula (1) is obtained through the above-described method.

The fluorine-containing compound of the present embodiment represented by Formula (2) can be produced using, for example, a production method shown below.

First, a fluorine-containing compound having a group corresponding to R⁴, X², and X³ in the fluorine-containing compound represented by Formula (2) is synthesized. Next, this fluorine-containing compound is cyclized to synthesize a nitrone having a pyrrole ring skeleton. Then, the resulting nitrone having a pyrrole ring skeleton and a Grignard reagent having a group corresponding to R⁵ are subjected to a Grignard reaction to introduce R⁵ in the fluorine-containing compound represented by Formula (2) into the 5-position of the pyrrole ring and synthesize a second intermediate compound in which a hydroxyl group is bound to a nitrogen atom at the 1-position.

Thereafter, in the same manner as in the case of the production of the fluorine-containing compound represented by Formula (1), the hydroxyl group bound to the nitrogen atom at the 1-position of the pyrrole ring of the second intermediate compound is converted into a nitroxide radical placed at the 1-position of the pyrrolidine ring.

The fluorine-containing compound represented by Formula (2) is obtained through the above-described method.

As an example of the method for producing the fluorine-containing compound of the present embodiment represented by Formula (2), a method for producing a fluorine-containing compound represented by Formula (24) will be described, for example.

4-(trifluoromethyl) benzaldehyde is dissolved in diethyl ether (Et₂O) and cooled to −20° C. Tetrahydrofuran (THF) containing vinylmagnesium bromide is added dropwise to this solution to cause a reaction, and allyl alcohol represented by Formula (2-1) is obtained.

Next, allyl alcohol represented by Formula (2-1) is dissolved in dichloromethane and oxidized using Dess-Martin periodinane to obtain vinyl ketone represented by Formula (2-2).

Next, vinyl ketone represented by Formula (2-2), 1-nitroethyl-4-(trifluoromethyl)benzene, molecular sieves (MS4A), and tetrahydrofuran (THF) are mixed with each other and stirred. A tetrahydrofuran solution containing tetrabutylammonium fluoride (TBAF) is added dropwise to this mixture to synthesize a fluorine-containing compound represented by Formula (2-3).

Next, water and ammonium chloride are added to the fluorine-containing compound represented by Formula (2-3) and cooled in an ice bath, zinc is added thereto, and cyclization is performed by causing a reaction while raising the temperature to room temperature to synthesize a nitrone represented by Formula (2-4). Then, tetrahydrofuran (THF) containing methylmagnesium bromide which is a Grignard reagent is added dropwise to the nitrone represented by Formula (2-4) to cause an addition reaction, and hydroxyamine represented by Formula (2-5) is obtained.

Thereafter, hydroxyamine represented by Formula (2-5) is dissolved in methanol (MeOH), aqueous ammonia and copper acetate monohydrate (Cu(OAc)₂) are added thereto to form a reaction solution, and an oxidation reaction is caused while blowing oxygen gas.

The fluorine-containing compound represented by Formula (24) is obtained through the above-described process.

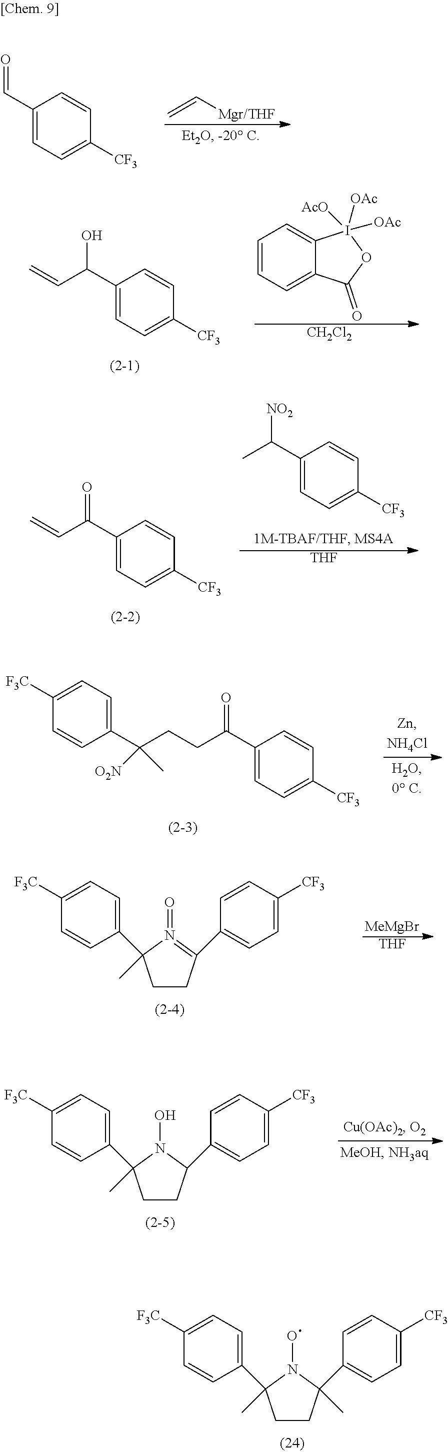

### “Contrast Medium”

A contrast medium of the present embodiment contains a fluorine-containing compound of the present embodiment. The contrast medium of the present embodiment is a contrast medium for magnetic resonance imaging diagnosis using fluorine as a detection nucleus.

The contrast medium of the present embodiment can be produced, for example, through a method for formulating the fluorine-containing compound of the present embodiment into forms such as a solid formulation, a powder preparation, and a liquid formulation using a well-known formulation technique.

The contrast medium of the present embodiment may contain one kind or two or more kinds of additives, such as an excipient, a stabilizer, a surfactant, a buffer agent, and an electrolyte, used in well-known formulations as necessary in addition to the fluorine-containing compound of the present embodiment.

Since the contrast medium of the present embodiment contains the fluorine-containing compound of the present invention, it is highly stable in vivo. In addition, the contrast medium of the present embodiment is used to obtain a high-sensitivity magnetic resonance image using fluorine as a detection nucleus.

The embodiment of the present invention has been described. However, each configuration and combination thereof in each embodiment are merely an example, and addition, omission, replacement, and other modifications of the configuration can be made within the scope not departing from the gist of the present invention.

### EXAMPLES

### Example 1

### (Synthesis of Compound 11)

<Synthesis of 1-(2-(trifluoromethyl)phenyl)-2-propene-1-ol (1-12)>

In an argon stream, 12.5 mL (95 mmol) of 2-(trifluoromethyl) benzaldehyde was dissolved in 100 mL of diethyl ether (Et₂O) and cooled to −20° C. 100 mL of tetrahydrofuran (THF) containing 100 mmol of vinylmagnesium bromide is added dropwise to this solution over 30 minutes and stirred at −20° C. for 14 hours to cause a reaction.

After the temperature of the reaction solution was raised to room temperature, a saturated aqueous ammonium chloride solution was added thereto, extraction was performed with diethyl ether, and the extract was dried with magnesium sulfate. Thereafter, the dried extract was concentrated under reduced pressure, and a colorless liquid of 1-(2-(trifluoromethyl)phenyl)-2-propene-1-ol which was a target product and represented by Formula (1-12) below was obtained (yield of 19.068 g, percent yield of 99%).

<Synthesis of 1-(2-(trifluoromethyl)phenyl)-2-propene-1-one (1-13)>

10.817 g (53.5 mmol) of the synthesized 1-(2-(trifluoromethyl)phenyl)-2-propene-1-ol (1-12) was dissolved in 200 mL of dichloromethane, and 25.000 g (58.9 mmol) of Dess-Martin periodinane was added thereto over 30 minutes. After stirring at room temperature for 2 hours, 200 mL of a saturated sodium hydrogen carbonate aqueous solution was added thereto over 20 minutes, followed by adding 100 mL of a saturated sodium hydrogen carbonate aqueous solution thereto and stirred at room temperature for 13 hours.

After the reaction solution was extracted with dichloromethane, it was dried with magnesium sulfate. After the dried reaction solution was concentrated under reduced pressure, the resulting crude product was purified through silica gel column chromatography (hexane:ethyl acetate=95:5 to 9:1) to obtain a colorless liquid of 1-(2-(trifluoromethyl)phenyl)-2-propene-1-one (yield of 8.864 g, percent yield of 83%) which was a target product and represented by Formula (1-13).

<Synthesis of 4-methyl-4-nitro-1-(2-(trifluoromethyl)phenyl)pentane-1-one (1-14)>

In an argon stream, 8.864 g (44.3 mmol) of 1-(2-(trifluoromethyl)phenyl)-2-propene-1-one (1-13) synthesized through the above-described reaction, 4.38 mL (48.7 mmol) of 2-nitropropane, 2.000 g of molecular sieves 4A (MS4A), and 25 mL of tetrahydrofuran (THF) were mixed with each other and stirred. 20 mL of a tetrahydrofuran solution containing 20.0 mmol of tetrabutylammonium fluoride (TBAF) was added dropwise to this mixture over 20 minutes and stirred at room temperature for 19 hours to cause a reaction.

The reaction solution was filtered with Celite, and then concentrated under reduced pressure. The resulting crude product was produced through silica gel column chromatography (hexane:ethyl acetate=95:5) to obtain a yellow liquid of 4-methyl-4-nitro-1-(2-(trifluoromethyl)phenyl)pentane-1-one (yield of 5.515 g, percent yield of 43%) which was a target product and represented by Formula (1-14).

<Synthesis of 2,2-dimethyl-5-(2-(trifluoromethyl)phenyl)-3,4-dihydro-2H-pyrrole-1-oxide (1-15)>

25 mL of water and 1.022 g (19.1 mmol) of ammonium chloride were added to 5.515 g (19.1 mmol) of 4-methyl-4-nitro-1-(2-(trifluoromethyl)phenyl)pentane-1-one (1-14), which was synthesized through the above-described reaction, and cooled in an ice bath. 3.746 g (57.3 mmol) of zinc was gradually added thereto over 30 minutes and stirred for 5 hours while raising the temperature to room temperature to cause a reaction.

The reaction solution was filtered with Celite, and then concentrated under reduced pressure. Chloroform was added to the resulting concentrate and dried with magnesium sulfate. The magnesium sulfate was filtered off and then concentrated under reduced pressure, and a brown liquid of 2,2-dimethyl-5-(2-(trifluoromethyl)phenyl)-3,4-dihydro-2H-pyrrole-1-oxide which was a target product and represented by Formula (1-15) was obtained (yield of 4.888 g, percent yield of 99%).

<Synthesis of 2,2,5-trimethyl-5-(2-(trifluoromethyl)phenyl)pyrrolidine-1-oxyl (11)>

In an argon stream, 10 mL of a tetrahydrofuran solution containing 18.3 mmol of 2,2-dimethyl-5-(2-(trifluoromethyl)phenyl)-3,4-dihydro-2H-pyrrole-1-oxide (1-15) synthesized through the above-described reaction was added dropwise to 27.5 mL of a tetrahydrofuran solution containing 27.5 mmol of methylmagnesium bromide, which was a Grignard reagent cooled in an ice bath, over 10 minutes and stirred at 60° C. for 21 hours to cause a reaction.

After the temperature of the reaction solution was cooled to room temperature, a saturated aqueous ammonium chloride solution was added to the reaction solution, and extraction was performed with diethyl ether. After the organic layer was washed with water and then washed with saturated saline, it was dried with magnesium sulfate and concentrated under reduced pressure.

The resulting concentrate was dissolved in 25 mL of methanol, 2.5 mL of 28% aqueous ammonia and 0.750 g (3.76 mmol) of copper acetate monohydrate (Cu(OAc)₂·H₂O) were added thereto to prepare a reaction solution and stirred for 1 hour while blowing oxygen gas to cause a reaction.

The reaction solution was extracted with chloroform, dried with magnesium sulfate, and concentrated under reduced pressure. The resulting crude product was purified through silica gel column chromatography (hexane:ethyl acetate=95:5) to obtain an orange liquid of 2,2,5-trimethyl-5-(2-(trifluoromethyl)phenyl)pyrrolidine-1-oxyl (yield of 0.211 g, percent yield of 4%) which was a target product and represented by Formula (11).

When the resulting compound was subjected to mass spectrometry, a peak was confirmed at m/z=272 (M⁺). From this, it was confirmed that the synthesized compound is a compound represented by Formula (11). In addition, the purity of the compound represented by Formula (11) which was conformed through high-performance liquid chromatography (HPLC) was 95.0%.

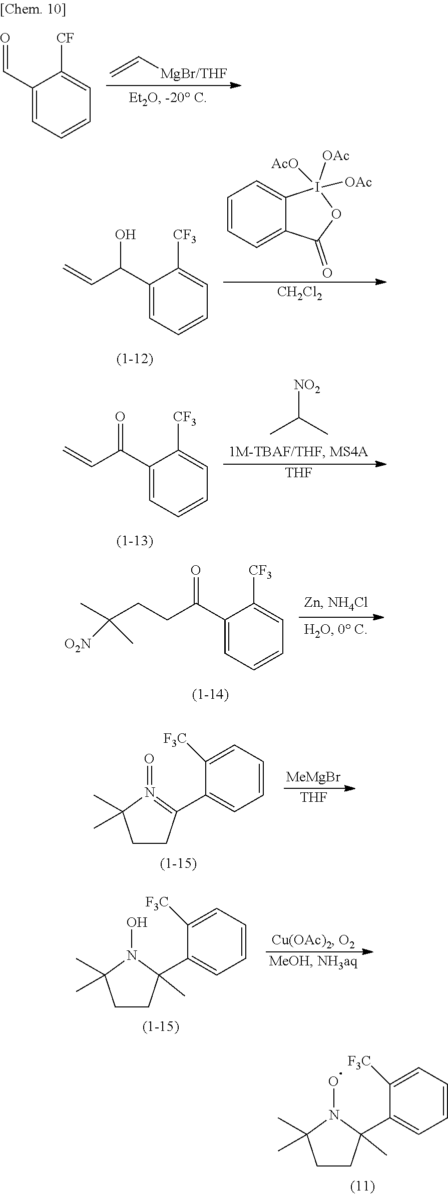

### Example 2

### (Synthesis of Compound 12)

<Synthesis of 5-methyl-5-nitrohexane-2-one (1-1)>

In an argon stream, 17.5 mL (210 mmol) of methyl vinyl ketone, 18.0 mL (200 mmol) of 2-nitropropane, 4.000 g of molecular sieves 4A (MS4A), and 90 mL of tetrahydrofuran (THF) were mixed with each other and stirred. 86 mL of a tetrahydrofuran solution containing 86 mmol of tetrabutylammonium fluoride (TBAF) was added dropwise to this mixture over 1 hour and stirred at room temperature for 18 hours to cause a reaction.

The reaction solution was filtered with Celite, and then concentrated under reduced pressure. The resulting crude product was purified through silica gel column chromatography (hexane:ethyl acetate=95:5 to 4:1) to obtain a yellow liquid of 5-methyl-5-nitrohexane-2-one which was a target product and represented by Formula (1-1) (yield of 25.213 g, percent yield of 79%).

<Synthesis of 2,2,5-trimethyl-3,4-dihydro-2H-pyrrole-1-oxide (1-2)>

100 mL of water and 8.895 g (166 mmol) of ammonium chloride were added to 25.213 g (158 mmol) of 5-methyl-5-nitrohexane-2-one (1-1), which was synthesized through the above-described reaction, and cooled in an ice bath. 31.056 g (475 mmol) of zinc was gradually added thereto over 50 minutes and stirred for 18 hours while raising the temperature to room temperature to cause a reaction.

The reaction solution was filtered with Celite, and then concentrated under reduced pressure. Chloroform was added to the resulting concentrate and dried with magnesium sulfate. The magnesium sulfate was filtered off and then concentrated under reduced pressure, and a brown liquid of 2,2,5-trimethyl-3,4-dihydro-2H-pyrrole-1-oxide which was a target product and represented by Formula (1-2) was obtained (yield of 19.577 g, percent yield of 97%).

<Synthesis of 2,2,5-trimethyl-5-(3-(trifluoromethyl)phenyl)pyrrolidine-1-oxyl (12)>

In an argon stream, 10 mL of a tetrahydrofuran solution containing 20.0 mmol of 2,2,5-trimethyl-3,4-dihydro-2H-pyrrole-1-oxide (1-2) synthesized through the above-described reaction was added dropwise to 30 mL of a tetrahydrofuran (THF) solution containing 30.0 mmol of 3-(trifluoromethyl)phenylmagnesium bromide, which was a Grignard reagent cooled in an ice bath, over 10 minutes and stirred at 60° C. for 15 hours to cause a reaction.

After the temperature of the reaction solution was cooled to room temperature, a saturated aqueous ammonium chloride solution was added to the reaction solution, and extraction was performed with diethyl ether. After the organic layer was washed with water and then washed with saturated saline, it was dried with magnesium sulfate and concentrated under reduced pressure.

The resulting concentrate was dissolved in 10 mL of methanol, 1.0 mL of 28% aqueous ammonia and 0.799 g (4.00 mmol) of copper acetate monohydrate (Cu(OAc)₂·H₂O) were added thereto to prepare a reaction solution and stirred for 1 hour while blowing oxygen gas to cause a reaction.

The reaction solution was extracted with chloroform, dried with magnesium sulfate, and concentrated under reduced pressure. The resulting crude product was purified through silica gel column chromatography (hexane:ethyl acetate=95:5) to obtain an orange liquid of 2,2,5-trimethyl-5-(3-(trifluoromethyl)phenyl)pyrrolidine-1-oxyl (yield of 1.190 g, percent yield of 21%) which was a target product and represented by Formula (12).

When the resulting compound was subjected to mass spectrometry, a peak was confirmed at m/z=272 (M⁺). From this, it was confirmed that the synthesized compound is a compound represented by Formula (12). In addition, the purity of the compound represented by Formula (12) which was conformed through high-performance liquid chromatography (HPLC) was 97.5%.

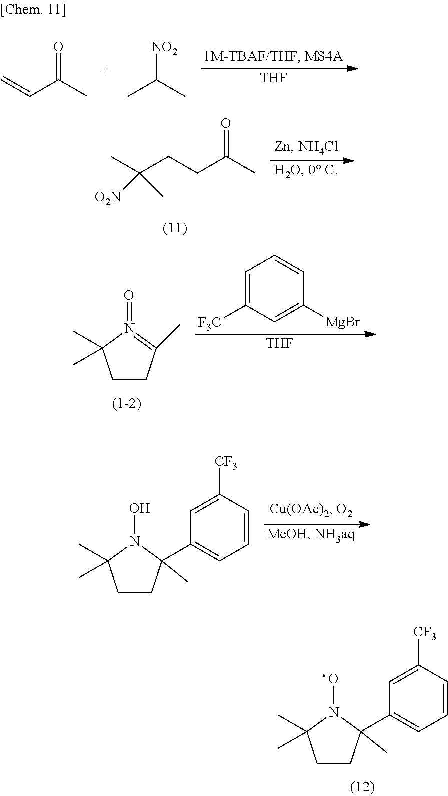

### Example 3

### (Synthesis of Compound 13)

<Synthesis of 2,2,5-trimethyl-5-(3,5-bis(trifluoromethyl)phenyl)pyrrolidine-1-oxyl (13)>

2,2,5-trimethyl-5-(3,5-bis(trifluoromethyl)phenyl)pyrrolidine-1-oxyl which was a target product and represented by Formula (13) was synthesized (yield of 1.128 g, percent yield of 17%) in the same manner as in Example 2 except that 3,5-bis(trifluoromethyl)phenylmagnesium bromide which was a Grignard reagent was used instead of 3-(trifluoromethyl)phenylmagnesium bromide.

When the resulting compound was subjected to mass spectrometry, a peak was confirmed at m/z=340 (M⁺). From this, it was confirmed that the synthesized compound is a compound represented by Formula (13). In addition, the purity of the compound represented by Formula (13) which was conformed through high-performance liquid chromatography (HPLC) was 95.2%.

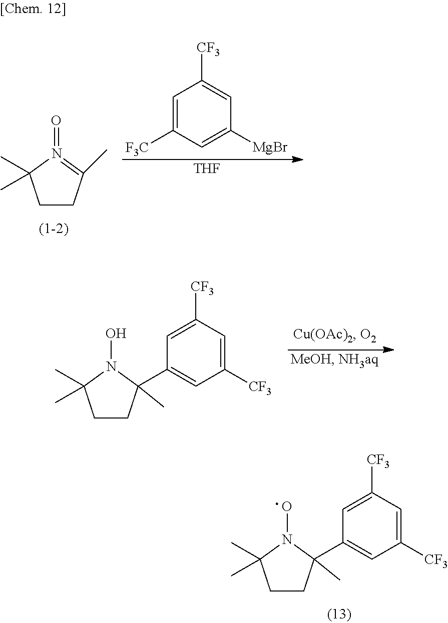

### Example 4

### (Synthesis of Compound 14)

<Synthesis of 2,2,5-trimethyl-5-(4-(trifluoromethyl)phenyl)pyrrolidine-1-oxyl (14)>

2,2,5-trimethyl-5-(4-(trifluoromethyl)phenyl)pyrrolidine-1-oxyl which was a target product and represented by Formula (14) was synthesized (yield of 1.248 g, percent yield of 23%) in the same manner as in Example 2 except that 4-(trifluoromethyl)phenylmagnesium bromide which was a Grignard reagent was used instead of 3-(trifluoromethyl)phenylmagnesium bromide.

When the resulting compound was subjected to mass spectrometry, a peak was confirmed at m/z=272 (M⁺). From this, it was confirmed that the synthesized compound is a compound represented by Formula (14). In addition, the purity of the compound represented by Formula (14) which was conformed through high-performance liquid chromatography (HPLC) was 96.8%.

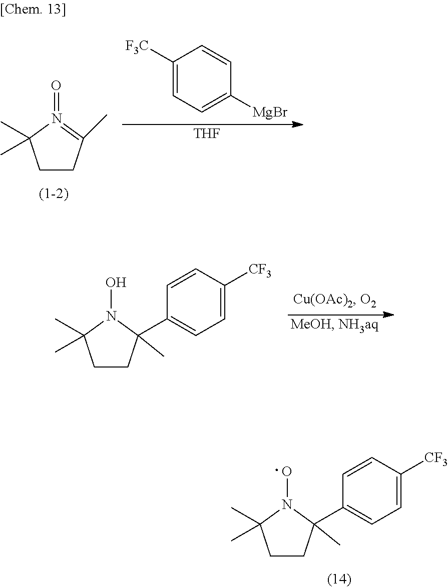

### Example 5

### (Synthesis of Compound 15)

<Synthesis of 1-bromo-3-((1,1,1,3,3,3-hexafluoro-2-(trifluoromethyl)propane-2-yl)oxy)benzene (1-4)>

The compound represented by Formula (1-4) was synthesized through a method described in Org. Lett., 2019, 21, 5206.

In an argon stream, 6.34 mL (50.0 mmol) of 1-bromo-3-iodobenzene was dissolved in 70 mL of dichloromethane and 70 mL of trifluoroethanol and cooled to −15° C. 13.540 g (51.0 mmol) of 65% metachlorobenzoic acid (mCPBA) was added thereto and stirred for 10 minutes, and then, 10.462 g (55.0 mmol) of p-toluenesulfonic acid monohydrate (TsOHH₂O) was added thereto at one time. The temperature of the reaction solution was raised to 40° C. over 1.5 hours, and the reaction solution was stirred for 1 hour. 12.614 g (75.0 mmol) of 1,3,5-trimethoxybenzene was added thereto at −15° C. and stirred for 10 minutes, and then concentrated under reduced pressure. The resulting concentrate was reprecipitated with methanol-diethyl ether to obtain a white solid of a diaryliodonium salt (1-3) (yield of 31.060 g, percent yield of 100%).

In an argon stream, 16.796 g (27.0 mmol) of the diaryliodonium salt (1-3) obtained through the above-described reaction and 9.39 mL (67.6 mmol) of nonafluoro-tert-butanol were added to a solution of 12.304 g (81.0 mmol) of cesium fluoride in toluene (50 mL) and stirred at 110° C. for 12 hours. After cooling to room temperature, water was added to the reaction solution, extraction was performed with diethyl ether, and the extract was dried with magnesium sulfate.

After the extract was concentrated under reduced pressure, the resulting crude product was purified through silica gel column chromatography (hexane) to obtain a white solid of 1-bromo-3-((1,1,1,3,3,3-hexafluoro-2-(trifluoromethyl)propane-2-yl)oxy)benzene which was a target product and represented by Formula (1-4) (yield of 6.611 g, percent yield of 63%).

<Synthesis of 2-(3-((1,1,1,3,3,3-hexafluoro-2-(trifluoromethyl)propane-2-yl)oxy)phenyl)-2,5,5-trimeth ylpyrrolidine-1-oxyl (15)>

1-bromo-3-((1,1,1,3,3,3-hexafluoro-2-(trifluoromethyl)propane-2-yl)oxy)benzene (1-4) obtained through the above-described reaction was dissolved in tetrahydrofuran (THF), the solution was added to a tetrahydrofuran solution to which iodine and metal magnesium were added to cause a reaction, and (3-((1,1,1,3,3,3-hexafluoro-2-(trifluoromethyl)propane-2-yl)oxy)phenyl)magnesium bromide which was a Grignard reagent was produced.

Then, 2-(3-((1,1,1,3,3,3-hexafluoro-2-(trifluoromethyl)propane-2-yl)oxy)phenyl)-2,5,5-trimeth ylpyrrolidine-1-oxyl which was a target product and represented by Formula (15) was synthesized (yield of 1.306 g, percent yield of 15%) in the same manner as in Example 2 except that (3-((1,1,1,3,3,3-hexafluoro-2-(trifluoromethyl)propane-2-yl)oxy)phenyl)magnesium bromide was used instead of 3-(trifluoromethyl)phenylmagnesium bromide.

When the resulting compound was subjected to mass spectrometry, a peak was confirmed at m/z=438 (M⁺). From this, it was confirmed that the synthesized compound is a compound represented by Formula (15). In addition, the purity of the compound represented by Formula (15) which was conformed through high-performance liquid chromatography (HPLC) was 96.3%.

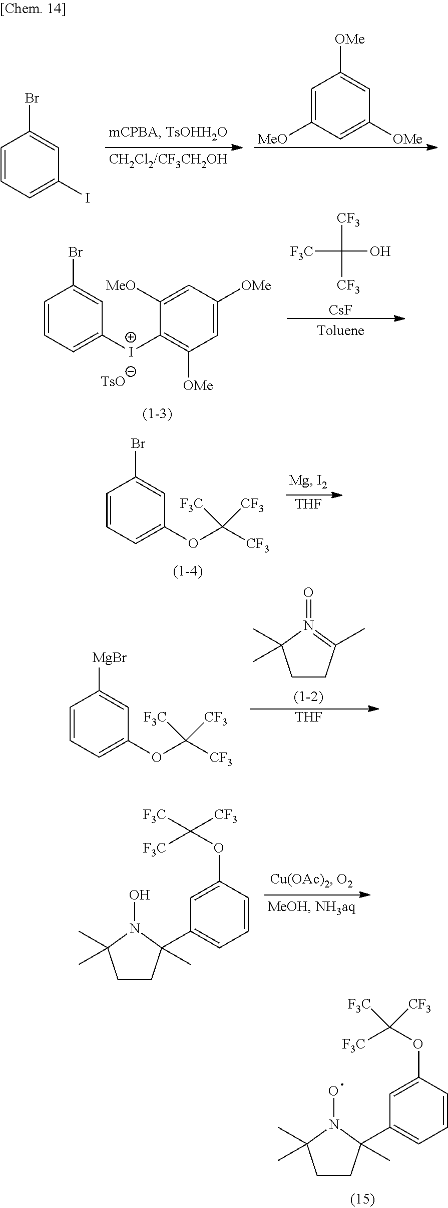

### Example 6

### (Synthesis of Compound 16)

<Synthesis of 2-(4-((1,1,1,3,3,3-hexafluoro-2-(trifluoromethyl)propane-2-yl)oxy)phenyl)-2,5,5-trimeth ylpyrrolidine-1-oxyl (16)>

1-bromo-4-((1,1,1,3,3,3-hexafluoro-2-(trifluoromethyl)propane-2-yl)oxy)benzene represented by Formula (1-5) was synthesized in the same manner as the compound represented by Formula (1-4) in Example 5 except that 1-bromo-4-iodobenzene was used as a starting material instead of 1-bromo-3-iodobenzene. Then, (4-((1,1,1,3,3,3-hexafluoro-2-(trifluoromethyl)propane-2-yl)oxy)phenyl)magnesium bromide which was a Grignard reagent was prepared in the same manner as in Example 5 using the compound represented by Formula (1-5) instead of the compound represented by Formula (1-4) in Example 5.

2-(4-((1,1,1,3,3,3-hexafluoro-2-(trifluoromethyl)propane-2-yl)oxy)phenyl)-2,5,5-trimethylpyrrolidine-1-oxyl which was a target product and represented by Formula (16) was synthesized (yield of 0.384 g, percent yield of 5%) in the same manner as in Example 2 except that (4-((1,1,1,3,3,3-hexafluoro-2-(trifluoromethyl)propane-2-yl)oxy)phenyl)magnesium bromide was used instead of 3-(trifluoromethyl)phenylmagnesium bromide.

When the resulting compound was subjected to mass spectrometry, a peak was confirmed at m/z=438 (M⁺). From this, it was confirmed that the synthesized compound is a compound represented by Formula (16). In addition, the purity of the compound represented by Formula (16) which was conformed through high-performance liquid chromatography (HPLC) was 96.3%.

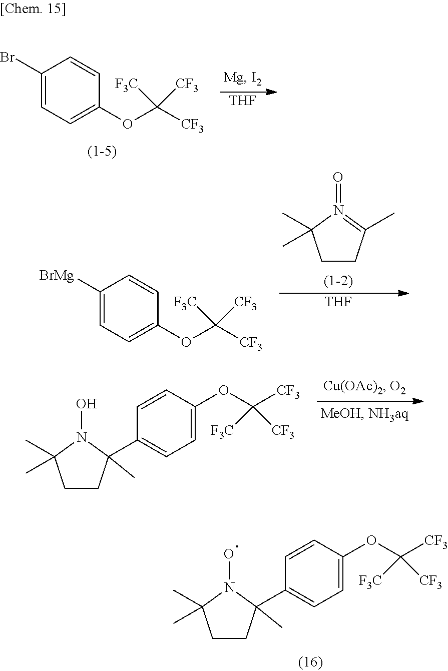

### Example 7

### (Synthesis of Compound 17)

<Synthesis of sodium-nonafluoro-tert-butoxide (1-6)>

In an argon stream, 14.6 mL (105 mmol) of nonafluoro-tert-butanol was added dropwise to a suspension of 4.364 g (100 mmol) of 55% sodium hydride in diethyl ether (Et₂O) (120 mL), which was cooled in an ice bath over 15 minutes, and stirred at room temperature for 2.5 hours to cause a reaction.

After concentrating the reaction solution under reduced pressure, hexane was added thereto, a produced white precipitate was filtered off and washed with hexane to obtain sodium-nonafluoro-tert-butoxide (yield of 25.800 g, percent yield of 100%) which was a target product and represented by Formula (1-6).

<Synthesis of 1-bromo-3-(((1,1,1,3,3,3-hexafluoro-2-(trifluoromethyl)propane-2-yl)oxy)methyl)benzene (1-7)>

In an argon stream, 10.569 g (41.0 mmol) of sodium-nonafluoro-tert-butoxide (1-6) obtained through the above-described reaction and 9.747 g (39.0 mmol) of 1-bromo-3-(bromomethyl)benzene were dissolved in 30 mL of dimethylformamide (DMF) and stirred at room temperature for 15 hours and then at 110° C. for 1 hour to cause a reaction.

After cooling the reaction solution to room temperature, water was added to the reaction solution, extraction was performed with chloroform, and the extract was dried with magnesium sulfate and concentrated under reduced pressure. The resulting crude product was purified through silica gel column chromatography (hexane) to obtain 1-bromo-3-(((1,1,1,3,3,3-hexafluoro-2-(trifluoromethyl)propane-2-yl)oxy)methyl)benzene which was a target product and represented by Formula (1-7) (yield of 12.827 g, percent yield of 81%).

<Synthesis of 2-(3-((1,1,1,3,3,3-hexafluoro-2-(trifluoromethyl)propane-2-yl)oxy)methyl)phenyl)-2,2,5-trimethylpyrrolidine-1-oxyl (17)>

Then, 3-(((1,1,1,3,3,3-hexafluoro-2-(trifluoromethyl)propane-2-yl)oxy)methyl)phenyl)magnesium bromide which was a Grignard reagent was prepared in the same manner as in Example 5 using the compound represented by Formula (1-7) instead of the compound represented by Formula (1-4) in Example 5.

2-(3-((1,1,1,3,3,3-hexafluoro-2-(trifluoromethyl)propane-2-yl)oxy)methyl)phenyl)-2,2,5-trimethylpyrrolidine-1-oxyl which was a target product and represented by Formula (17) was synthesized (yield of 2.058 g, percent yield of 20%) in the same manner as in Example 2 except that 3-(((1,1,1,3,3,3-hexafluoro-2-(trifluoromethyl)propane-2-yl)oxy)methyl)phenyl)magnesium bromide was used instead of 3-(trifluoromethyl)phenylmagnesium bromide.

When the resulting compound was subjected to mass spectrometry, a peak was confirmed at m/z=452 (M′). From this, it was confirmed that the synthesized compound is a compound represented by Formula (17). In addition, the purity of the compound represented by Formula (17) which was conformed through high-performance liquid chromatography (HPLC) was 94.4%.

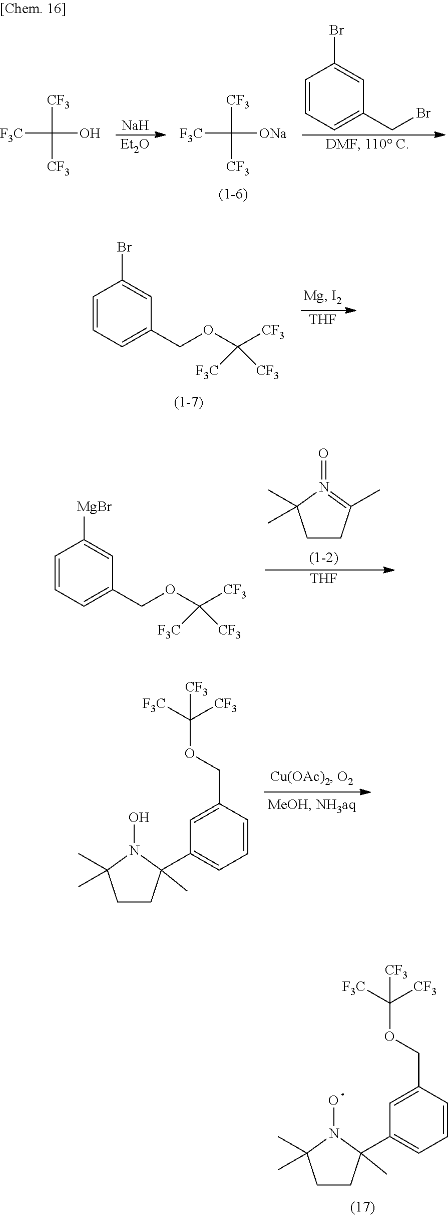

### Example 8

### (Synthesis of Compound 18)

<Synthesis of 2,2,5-trimethyl-5-(4,4,4-trifluorobutyl)pyrrolidine-1-oxyl (18)>

2,2,5-trimethyl-5-(4,4,4-trifluorobutyl)pyrrolidine-1-oxyl which was a target product and represented by Formula (18) was synthesized (yield of 0.220 g, percent yield of 4%) in the same manner as in Example 2 except that 4,4,4-trifluorobutylmagnesium bromide was used instead of 3-(trifluoromethyl)phenylmagnesium bromide.

When the resulting compound was subjected to mass spectrometry, a peak was confirmed at m/z=238 (M⁺). From this, it was confirmed that the synthesized compound is a compound represented by Formula (18). In addition, the purity of the compound represented by Formula (18) which was conformed through high-performance liquid chromatography (HPLC) was 95.6%.

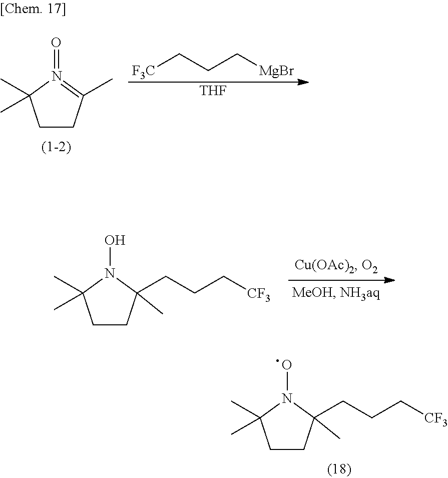

### Example 9

### (Synthesis of Compound 19)

<Synthesis of 2-(2-bromoethoxy)-1,1,1,3,3,3-hexafluoro-2-(trifluoromethyl)propane (1-8)>

In an argon stream, 12.385 g (48.0 mmol) of sodium-nonafluoro-tert-butoxide (1-6) obtained in the same manner as in Example 7 and 3.45 mL (40.0 mmol) of 1,2-dibromoethane were dissolved in 25 mL of dimethylformamide (DMF) and stirred at room temperature for 18 hours and then at 80° C. for 1 hour to cause a reaction.

After cooling the reaction solution to room temperature, water was added to the reaction solution, extraction was performed with diethyl ether, and the extract was dried with magnesium sulfate. The dried agent was filtered off and then distilled under normal pressure to obtain 2-(2-bromoethoxy)-1,1,1,3,3,3-hexafluoro-2-(trifluoromethyl)propane which was a target product and represented by Formula (1-8) (yield of 9.783 g, percent yield of 71%).

<Synthesis of 2-(2-((1,1,1,3,3,3-hexafluoro-2-(trifluoromethyl)propane-2-yl)oxy)ethyl)-2,2,5-trimethyl pyrrolidine-1-oxyl (19)>

2-(2-bromoethoxy)-1,1,1,3,3,3-hexafluoro-2-(trifluoromethyl)propane (1-8) obtained through the above-described reaction was dissolved in diethyl ether (Et₂O), the solution was added to a diethyl ether solution to which iodine and metal magnesium were added to cause a reaction, and a Grignard reagent was produced.

Then, 2-(2-((1,1,1,3,3,3-hexafluoro-2-(trifluoromethyl)propane-2-yl)oxy)ethyl)-2,2,5-trimethyl pyrrolidine-1-oxyl which was a target product and represented by Formula (19) was synthesized (yield of 0.780 g, percent yield of 10%) in the same manner as in Example 2 except that the Grignard reagent prepared from the compound represented by Formula (1-8) was used instead of 3-(trifluoromethyl)phenylmagnesium bromide.

When the resulting compound was subjected to mass spectrometry, a peak was confirmed at m/z=390 (M⁺). From this, it was confirmed that the synthesized compound is a compound represented by Formula (19). In addition, the purity of the compound represented by Formula (19) which was conformed through high-performance liquid chromatography (HPLC) was 96.8%.

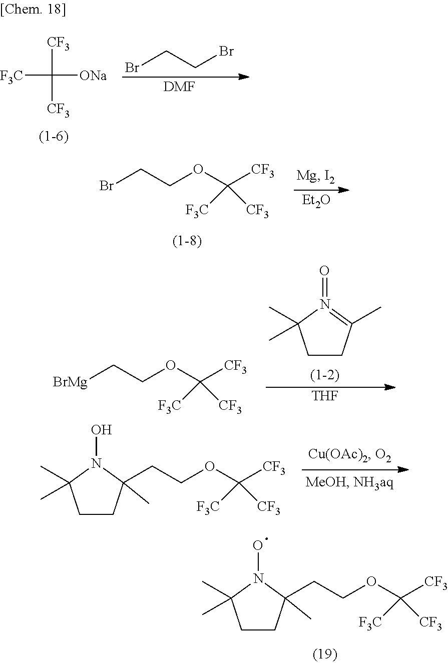

### Example 10

### (Synthesis of Compound 20)

<Synthesis of 3-nitropentane (1-9)>

In an argon stream, 15.00 g (99.3 mmol) of 3-bromopentane was added to a solution of 8.281 g (120.0 mmol) of sodium nitrite in dimethyl sulfoxide (DMSO) (80 mL) and stirred at 40° C. for 20 hours to cause a reaction.

After cooling the reaction solution to room temperature, ice water was added to the reaction solution, extraction was performed with pentane, and the extract was dried with magnesium sulfate. The dried agent was filtered off and then distilled under reduced pressure to obtain 3-nitropentane which was a target product and represented by Formula (1-9) (yield of 7.030 g, percent yield of 60%).

<Synthesis of 5-ethyl-5-nitroheptane-2-one (1-10)>

In an argon stream, 7.030 g (60.0 mmol) of 3-nitropentane (1-9) obtained through the above-described reaction, 5.26 mL (63.0 mmol) of methyl vinyl ketone, 2.000 g of molecular sieves 4A (MS4A), and 50 mL of tetrahydrofuran (THF) were mixed with each other and stirred. Then, 25 mL of a tetrahydrofuran solution containing 25.0 mmol of tetrabutylammonium fluoride (TBAF) was added dropwise thereto over 1 hour and stirred at room temperature for 18 hours to cause a reaction.

The reaction solution was filtered with Celite, and then concentrated under reduced pressure. The resulting crude product was purified through silica gel column chromatography (hexane:ethyl acetate=95:5 to 9:1) to obtain a yellow liquid of 5-ethyl-5-nitroheptane-2-one (1-10) which was a target product and represented by Formula (1-10) (yield of 7.980 g, percent yield of 71%).

<Synthesis of 2,2-diethyl-5-methyl-3,4-dihydro-2H-pyrrole-1-oxide (1-11)>

50 mL of water and 2.393 g (44.7 mmol) of ammonium chloride were added to 7.980 g (42.6 mmol) of 5-ethyl-5-nitroheptane-2-one (1-10), which was synthesized through the above-described reaction, and cooled in an ice bath. 11.141 g (170 mmol) of zinc was gradually added thereto over 50 minutes and stirred for 15 hours while raising the temperature to room temperature to cause a reaction.

The reaction solution was filtered with Celite, and then concentrated under reduced pressure. Chloroform was added to the resulting concentrate and dried with magnesium sulfate. The magnesium sulfate was filtered off and then concentrated under reduced pressure, and a brown liquid of 2,2-diethyl-5-methyl-3,4-dihydro-2H-pyrrole-1-oxide which was a target product and represented by Formula (1-11) was obtained (yield of 4.299 g, percent yield of 65%).

<Synthesis of 2,2-diethyl-5-methyl-5-(3-(trifluoromethyl)phenyl)pyrrolidine-1-oxyl (20)>

2,2-diethyl-5-methyl-5-(3-(trifluoromethyl)phenyl)pyrrolidine-1-oxyl which was a target product and represented by Formula (20) was synthesized (yield of 1.081 g, percent yield of 18%) in the same manner as in Example 2 except that 2,2-diethyl-5-methyl-3,4-dihydro-2H-pyrrole-1-oxide (1-11) synthesized through the above-described reaction was used instead of 2,2,5-trimethyl-3,4-dihydro-2H-pyrrole-1-oxide (1-2).

When the resulting compound was subjected to mass spectrometry, a peak was confirmed at m/z=300 (M⁺). From this, it was confirmed that the synthesized compound is a compound represented by Formula (20). In addition, the purity of the compound represented by Formula (20) which was conformed through high-performance liquid chromatography (HPLC) was 96.2%.

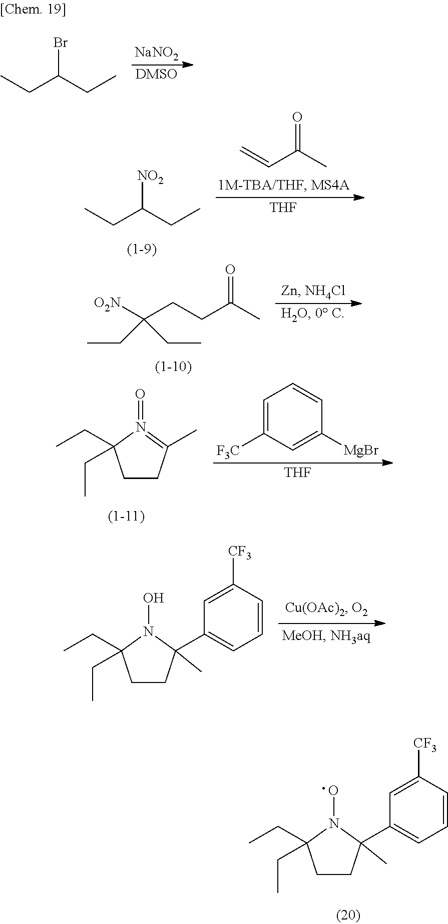

### Example 11

### (Synthesis of Compound 21)

<Synthesis of 2,2-diethyl-5-methyl-5-(4-(trifluoromethyl)phenyl)pyrrolidine-1-oxyl (21)>

2,2-diethyl-5-methyl-5-(4-(trifluoromethyl)phenyl)pyrrolidine-1-oxyl which was a target product and represented by Formula (21) was synthesized (yield of 1.442 g, percent yield of 24%) in the same manner as in Example 10 except that 4-(trifluoromethyl)phenylmagnesium bromide was used instead of 3-(trifluoromethyl)phenylmagnesium bromide.

When the resulting compound was subjected to mass spectrometry, a peak was confirmed at m/z=300 (M⁺). From this, it was confirmed that the synthesized compound is a compound represented by Formula (21). In addition, the purity of the compound represented by Formula (21) which was conformed through high-performance liquid chromatography (HPLC) was 96.8%.

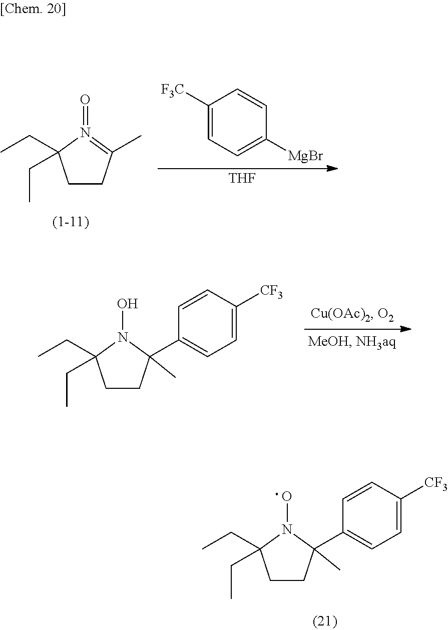

### Example 12

### (Synthesis of Compound 22)

<Synthesis of 2,2-diethyl-5-(4-((1,1,1,3,3,3-hexafluoro-2-(trifluoromethyl)propane-2-yl)oxy)phenyl)-5-methylpyrrolidine-1-oxyl (22)>

2,2-diethyl-5-(4-((1,1,1,3,3,3-hexafluoro-2-(trifluoromethyl)propane-2-yl)oxy)phenyl)-5-methylpyrrolidine-1-oxyl which was a target product and represented by Formula (22) was synthesized (yield of 0.933 g, percent yield of 10%) in the same manner as in Example 6 except that 2,2-diethyl-5-methyl-3,4-dihydro-2H-pyrrole-1-oxide (1-11) synthesized in the same manner as in Example 10 was used instead of 2,2,5-trimethyl-3,4-dihydro-2H-pyrrole-1-oxide (1-2).

When the resulting compound was subjected to mass spectrometry, a peak was confirmed at m/z=466 (M⁺). From this, it was confirmed that the synthesized compound is a compound represented by Formula (22). In addition, the purity of the compound represented by Formula (22) which was conformed through high-performance liquid chromatography (HPLC) was 97.1%.

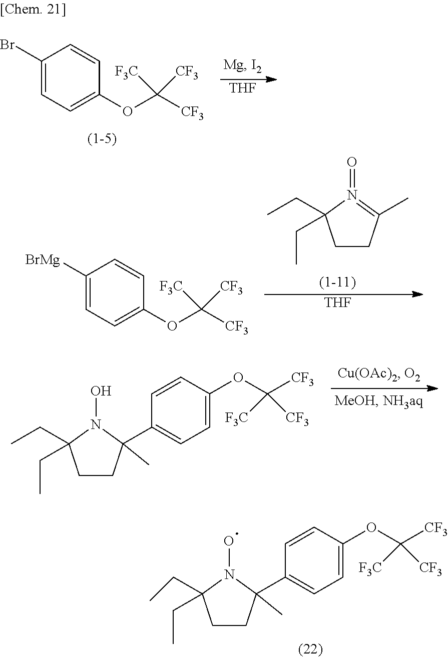

### Example 13

### (Synthesis of Compound 23)

<Synthesis of 2,2-diethyl-5-(2-((1,1,1,3,3,3-hexafluoro-2-(trifluoromethyl)propane-2-yl)oxy)ethyl)-5-m ethylpyrrolidine-1-oxyl (23)>

2,2-diethyl-5-(2-((1,1,1,3,3,3-hexafluoro-2-(trifluoromethyl)propane-2-yl)oxy)ethyl)-5-methylpyrrolidine-1-oxyl which was a target product and represented by Formula (23) was synthesized (yield of 0.920 g, percent yield of 11%) in the same manner as in Example 9 except that 2,2-diethyl-5-methyl-3,4-dihydro-2H-pyrrole-1-oxide (1-11) synthesized in the same manner as in Example 10 was used instead of 2,2,5-trimethyl-3,4-dihydro-2H-pyrrole-1-oxide (1-2).

When the resulting compound was subjected to mass spectrometry, a peak was confirmed at m/z=418 (M⁺). From this, it was confirmed that the synthesized compound is a compound represented by Formula (23). In addition, the purity of the compound represented by Formula (23) which was conformed through high-performance liquid chromatography (HPLC) was 97.5%.

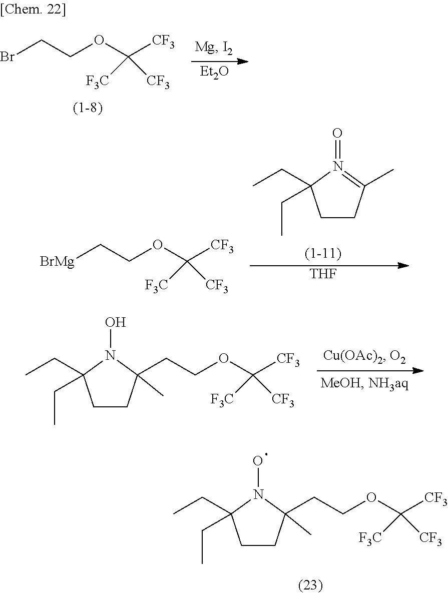

### Example 14

### (Synthesis of Compound 25)

<Synthesis of 2,2,5-trimethyl-5-(4-(2,2,2-trifluoroethoxy)phenyl)pyrrolidine-1-oxyl (25)>

2,2,5-trimethyl-5-(4-(2,2,2-trifluoroethoxy)phenyl)pyrrolidine-1-oxyl (25) which was a target product and represented by Formula (25) was synthesized (yield of 0.785 g, percent yield of 13%) in the same manner as in Example 2 except that (4-(2,2,2-trifluoroethoxy)phenyl)magnesium bromide which was a Grignard reagent was used instead of 3-(trifluoromethyl)phenylmagnesium bromide.

When the resulting compound was subjected to mass spectrometry, a peak was confirmed at m/z=302 (M⁺). From this, it was confirmed that the synthesized compound is a compound represented by Formula (25). In addition, the purity of the compound represented by Formula (25) which was conformed through high-performance liquid chromatography (HPLC) was 95.8%.

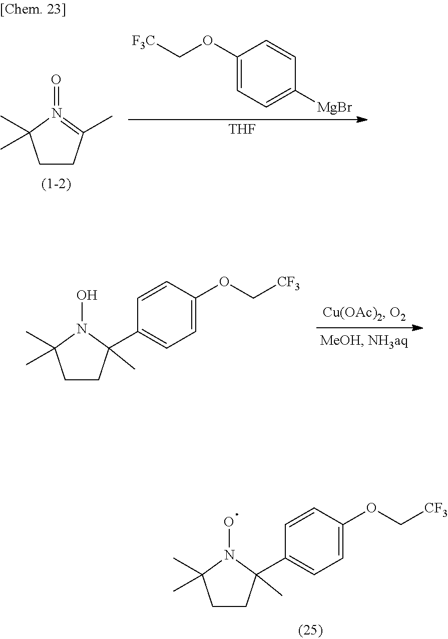

### Example 15

### (Synthesis of Compound 26)

<Synthesis of 2-(4-((1,1,1,3,3,3-hexafluoropropane-2-yl)oxy)phenyl)-2,5,5-trimethylpyrrolidine-1-oxyl (26)>

1-bromo-4-((1,1,1,3,3,3-hexafluoropropane-2-yl)oxy)benzene represented by Formula (1-16) was synthesized in the same manner as the compound represented by Formula (1-4) in Example 5 except that 1-bromo-4-iodobenzene was used as a starting material instead of 1-bromo-3-iodobenzene and 1,1,1,3,3,3-hexafluoro-2-propanol was used instead of nonafluoro-tert-butanol.

Then, ((4-((1,1,1,3,3,3-hexafluoro)propane-2-yl)oxy)phenyl)magnesium bromide which was a Grignard reagent was prepared in the same manner as in Example 5 using the compound represented by Formula (1-16) instead of the compound represented by Formula (1-4) in Example 5.

2-(4-((1,1,1,3,3,3-hexafluoropropane-2-yl)oxy)phenyl)-2,5,5-trimethylpyrrolidin e-1-oxyl which was a target product and represented by Formula (26) was synthesized (yield of 1.110 g, percent yield of 15%) in the same manner as in Example 2 except that ((4-((1,1,1,3,3,3-hexafluoro)propane-2-yl)oxy)phenyl)magnesium bromide was used instead of 3-(trifluoromethyl)phenylmagnesium bromide.

When the resulting compound was subjected to mass spectrometry, a peak was confirmed at m/z=370 (M⁺). From this, it was confirmed that the synthesized compound is a compound represented by Formula (26). In addition, the purity of the compound represented by Formula (26) which was conformed through high-performance liquid chromatography (HPLC) was 96.5%.

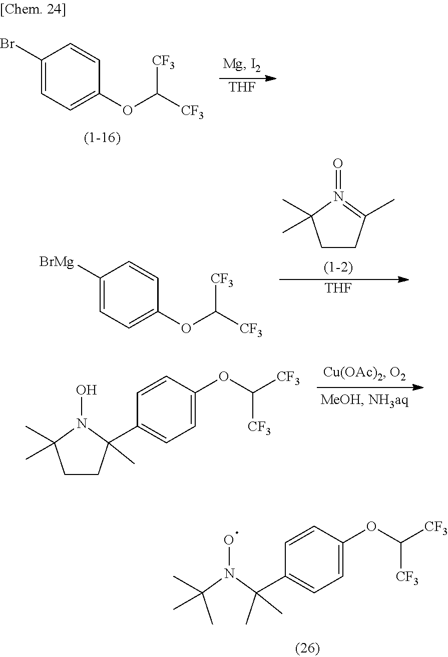

### Comparative Example 1

Trifluoromethylbenzene represented by Formula (A1) below was prepared.

### Comparative Example 2

### (Synthesis of Compound A2)

Trifluoromethylbenzene represented by Formula (A1) and 4-hydroxy-2,2,6,6-tetramethylpiperidine-1-oxyl represented by Formula (A2) below (manufactured by Tokyo Chemical Industry Co., Ltd.) were mixed with each other at a molar ratio ((A1):(A2)) of 1:1 to obtain a compound of Comparative Example 2.

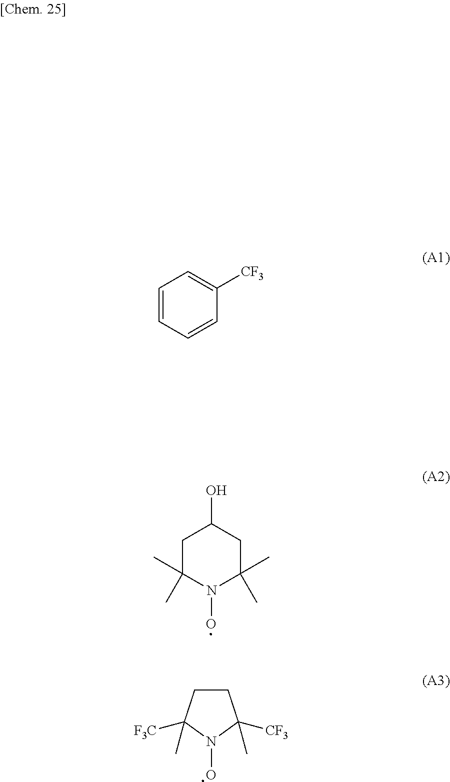

For the compounds of Examples 1 to 15 and Comparative Examples 1 and 2 thus obtained, the ¹⁹F spin-lattice relaxation time (T1) was measured through methods shown below. The results are shown in Table 1.

(Measurement of ¹⁹F spin-lattice relaxation time (T1)) A compound was dissolved in a deuterated chloroform solution at a concentration of 50 mM, and the longitudinal relaxation time (T1) of ¹⁹F nucleus was measured through an inversion recovery method using a 500 MHz NMR device under the conditions shown below.

(Measurement Conditions)
NMR device: JNM-ECA500 (manufactured by JEOL)
Measurement temperature: 36° C.
Pulse sequence: double_pulse
relaxation_delay: 10[s]
tau_interval: 4, 3, 2, 1, 0.8, 0.6, 0.4, 0.2, 0.1[s], 80, 60, 40, 20, 10, 8, 6, 4, 2 [ms]
Number of times of integration: 16 times

In addition, for the compounds of Examples 1 to 15 and the compound of Comparative Example 3 represented by Formula (A3) above, the energy level of each semi-occupied molecular orbital (SOMO) was calculated through the method shown below. The results are shown in Table 1.

### (Calculation of Energy Level of SOMO)

Gaussian 09 manufactured by Gaussian, Inc. was used to calculate molecular orbitals of the compounds. The energy level of each semi-occupied molecular orbital (SOMO) was calculated through a structure optimization calculation by density-functional theory (DFT) in which B3LYP was used as a functional and 6-31+G(d,p) was used as a basis function.

TABLE 1

| | | T1 | |
|---|---|---|---|
| | Compound | 50 mM/CDCl₃ (sec) | SOMO (eV) |
| Example 1 | 11 | 0.022 | −2.288 |
| Example 2 | 12 | 0.032 | −2.419 |
| Example 3 | 13 | 0.024 | −2.556 |
| Example 4 | 14 | 0.080 | −2.422 |
| Example 5 | 15 | 0.035 | −2.415 |
| Example 6 | 16 | 0.084 | −2.424 |
| Example 7 | 17 | 0.062 | −2.397 |
| Example 8 | 18 | 0.032 | −2.258 |
| Example 9 | 19 | 0.041 | −2.061 |
| Example 10 | 20 | 0.030 | −2.404 |
| Example 11 | 21 | 0.078 | −2.440 |
| Example 12 | 22 | 0.084 | −2.449 |
| Example 13 | 23 | 0.040 | −2.326 |
| Example 14 | 25 | 0.090 | −2.340 |
| Example 15 | 26 | 0.088 | −2.395 |
| Comparative | A1 | 2.240 | — |
| Example 1 | | | |
| Comparative | A1, A2 | 0.440 | — |
| Example 2 | | | |
| Comparative | A3 | — | −2.935 |
| Example 3 | | | |

As shown in Table 1, the compounds of Examples 1 to 15 had shorter ¹⁹F spin-lattice relaxation time (T1) than the compounds of Comparative Examples 1 and 2.

In addition, the compounds of Examples 1 to 15 had higher energy levels of semi-occupied molecular orbitals (SOMO) than the compound of Comparative Example 3.

In addition, for each of the compounds of Example 4, Example 6, and Comparative Example 1, a 5 mM deuterated chloroform solution and a 10 mM deuterated chloroform solution were prepared, and T1-weighted images (phantom images) were obtained under the following imaging conditions.

(Imaging Conditions)
Imaging device: MRI BioSpec117/11 (manufactured by Bruker)
Pulse sequence: RAREVTR
Repetition time: TR=1500 ms
Echo time: TE=12 ms
Number of times of integration: 36 times
Total imaging time: 16 minutes 33 seconds

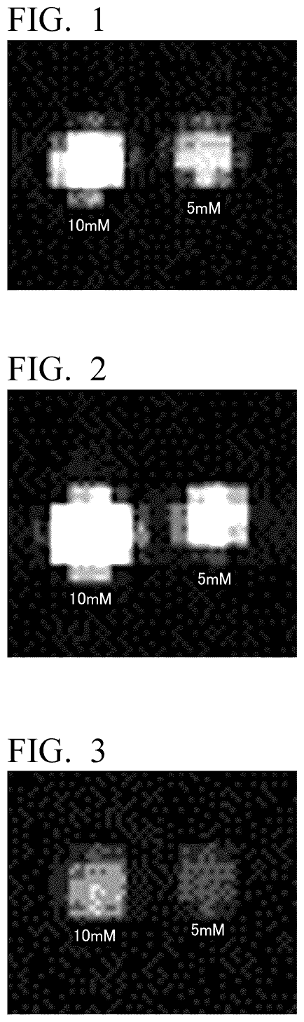

FIG. 1 is a T1-weighted ¹⁹F-MRI image of Example 4 (compound 14). FIG. 2 is a T1-weighted ¹⁹F-MRI image of Example 6 (compound 16). FIG. 3 is a T1-weighted ¹⁹F-MRI image of Comparative Example 1 (compound A1).

The image of Example 4 (compound 14) shown in FIG. 1 and the image of Example 6 (compound 16) shown in FIG. 2 were brighter than the image of Comparative Example 1 (compound A1) shown in FIG. 3 in either case of the 5 mM deuterated chloroform solution or the 10 mM deuterated chloroform solution.

In addition, it was possible to confirm from FIGS. 1 and 2 that sufficiently clinically applicable high-sensitivity images are obtained using Example 4 (compound 14) and Example 6 (compound 16) in a contrast medium for MRI diagnosis using fluorine as a detection nucleus.

### INDUSTRIAL APPLICABILITY

A contrast medium which is highly stable in vivo can be provided. In addition, a high-sensitivity magnetic resonance image can be obtained.

1. A fluorine-containing compound represented by General Formula (1) or General Formula (2) below, 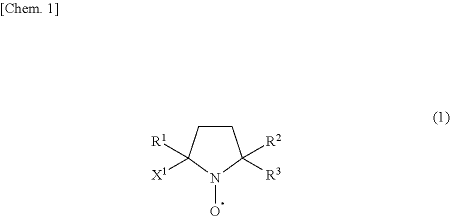
(in General Formula (1), R¹, R², and R³ each independently represent a C1-10 alkyl group unsubstituted or substituted with a substituent containing no fluorine atoms, and X¹ is a substituent represented by any of General Formulae (3-1) to (3-4)), 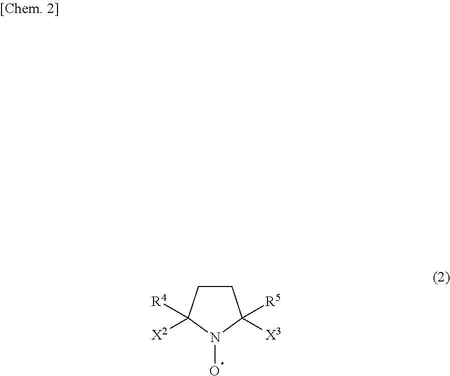
(in General Formula (2), R⁴ and R⁵ each independently represent a C1-10 alkyl group unsubstituted or substituted with a substituent containing no fluorine atoms, and X² and X³ are each independently a substituent represented by any of General Formulae (3-1) to (3-4)), 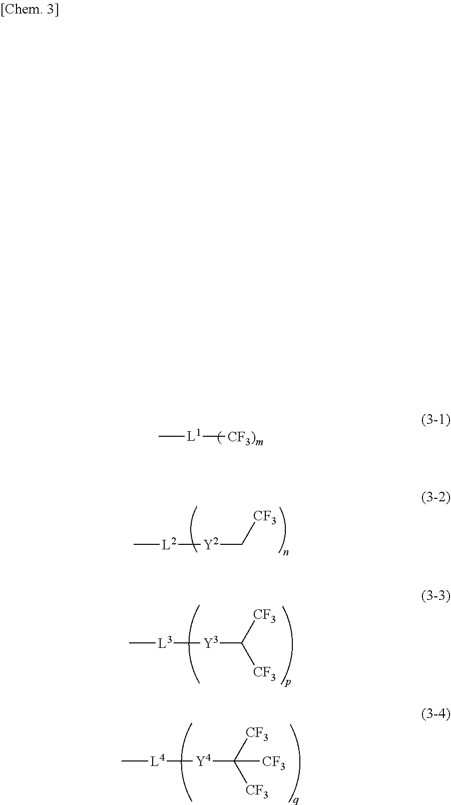
(in General Formula (3-1), L¹ is either a C1-10 chain hydrocarbon group unsubstituted or substituted with a substituent containing no fluorine atoms or a linking group containing a C6-12 aryl group unsubstituted or substituted with a substituent containing no fluorine atoms, and m is an integer of 1 to 5),
(in General Formula (3-2), L² is either a C1-10 chain hydrocarbon group unsubstituted or substituted with a substituent containing no fluorine atoms or a linking group containing a C6-12 aryl group unsubstituted or substituted with a substituent containing no fluorine atoms, Y² is an oxygen atom, and n is an integer of 1 to 5),
(in General Formula (3-3), L³ is either a C1-10 chain hydrocarbon group unsubstituted or substituted with a substituent containing no fluorine atoms or a linking group containing a C6-12 aryl group unsubstituted or substituted with a substituent containing no fluorine atoms, Y³ is an oxygen atom, and p is an integer of 1 to 5), and
(in General Formula (3-4), L⁴ is either a C1-10 chain hydrocarbon group unsubstituted or substituted with a substituent containing no fluorine atoms or a linking group containing a C6-12 aryl group unsubstituted or substituted with a substituent containing no fluorine atoms, Y⁴ is an oxygen atom, and q is an integer of 1 to 5).

2. The fluorine-containing compound according to claim 1,
wherein R¹, R², and R³ in General Formula (1) above each independently represent a C1-5 alkyl group unsubstituted or substituted with a substituent containing no fluorine atoms.

3. The fluorine-containing compound according to claim 1,
wherein R⁴ and R⁵ in General Formula (2) above each independently represent a C1-5 alkyl group unsubstituted or substituted with a substituent containing no fluorine atoms.

4. The fluorine-containing compound according to claim 1,
wherein, in General Formula (2) above, X² is the same as X³ and R⁴ is the same as R⁵.

5. The fluorine-containing compound according to claim 1,
wherein L¹ in General Formula (3-1), L² in General Formula (3-2), L³ in General Formula (3-3), and L⁴ in General Formula (3-4) are C1-5 chain hydrocarbon groups unsubstituted or substituted with a substituent containing no fluorine atoms.

6. The fluorine-containing compound according to claim 1,
wherein L¹ in General Formula (3-1), L² in General Formula (3-2), L³ in General Formula (3-3), and L⁴ in General Formula (3-4) are each a linking group containing a phenyl group or a biphenyl group.

7. The fluorine-containing compound according to claim 1,
wherein m in General Formula (3-1), n in General Formula (3-2), p in General Formula (3-3), and q in General Formula (3-4) are each 1 or 2.

8. The fluorine-containing compound according to claim 1, which is used in a contrast medium for magnetic resonance imaging diagnosis using fluorine as a detection nucleus.

9. A contrast medium for magnetic resonance imaging diagnosis using fluorine as a detection nucleus, comprising:
the fluorine-containing compound according to claim 1.
